(19) United States
(12) Patent Application Publication
(10) Pub. No.: US 2003/0186464 A1
(43) Pub. Date: Oct. 2, 2003

(54) Surface plasmon resonance methods

(76) Inventors: Michelle Arkin, San Francisco, CA (US); Jennifer Hyde, San Francisco, CA (US); Brian Cunningham, San Mateo, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/354,899

(22) Filed: Jan. 29, 2003

Related U.S. Application Data
(60) Provisional application No. 60/352,682, filed on Jan. 29, 2002.

(51) Int. Cl. G01N 33/543

(52) U.S. Cl. 436/518

(57) ABSTRACT

The present invention relates to the use of SPR methods to obtain information relating to molecular binding events. In one aspect of the present invention, methods are provided for determining whether a binding interaction is competitive or cooperative. In another aspect of the present invention, methods are provided for obtaining information where the binding interaction includes a formation of a reversible covalent bond.

### CROSS-REFERENCE TO RELATED APPLICATIONS

This application claims the benefit of U.S. Provisional Application Serial No. 60/352,682 filed on Jan. 29, 2002, which is incorporated herein by reference.

### BACKGROUND OF THE INVENTION

The use of surface plasmon resonance (“SPR”) to detect binding interactions has been described in the literature. SPR occurs when surface plasmon waves are excited at a metal/liquid interface. Light is directed at, and reflected from, the side of the surface not in contact with sample, and SPR causes a reduction in the reflected light intensity at a specific combination of angle and wavelength. Binding events cause changes in the refractive index at the surface layer, which are detected as changes in the SPR signal. In general, the refractive index change for a given change of mass concentration at the surface layer is practically the same for all proteins and peptides, and is similar for glycoproteins, lipids and nucleic acids.

BIACORE is a company that has commercialized the use of SPR to detect molecular binding events. Their products and general information can be found at their website, www.biacore.com.

As implemented by BIACORE, a gold surface is coated with a chemical matrix such as dextran. The substrate of interest, which can be for example, a protein, nucleic acid, lipid bilayer or ligand, is immobilized onto the matrix. Potential binding partners to the substrate of interest are then allowed to flow over the immobilized substrate surface and changes in refractive index are monitored. FIG. 1 is an illustration of the BIACORE SPR detection system.

In general, three types of data are obtained by such an SPR experiment. These data types will be illustrated using an immobilized protein and a soluble, small-molecule ligand (MW<1500, preferably MW<1000 and more preferably MW<750) as an example. The first type of information that can be obtained is the kinetics of association (binding) and of dissociation. As the ligand binds, the value of the refractive index increases in a time-dependent fashion. Similarly, as the bound ligand is eluted (typically with buffer), the value of the refractive index decreases in a time-dependent fashion. FIG. 2 is an illustrative example of an SPR experiment for measuring the kinetics of association and of dissociation.

The second and third types of information that can be obtained are the equilibrium binding affinity and the binding stoichiometry. The equilibrium binding affinity and stoichiometry are measured once a plateau or equilibrium binding is reached. The value of the refractive index at this plateau is related to: a) the amount of ligand added; b) the affinity of the ligand for the immobilized protein, and c) the stoichiometry of the binding interaction. In general, the affinity of the ligand is measured by monitoring the extent of binding at the plateau region at various ligand concentrations. FIG. 3 is an illustrative SPR experiment of the binding of three different compounds at 250 μM to interleukin-2 (“IL-2”).

SPR methods described in the prior art typically use a single immobilized substrate and a single soluble binding partner, and the binding event generally involves a noncovalent interaction. In addition, SPR has been used to examine noncovalent binding interactions to molecules containing intermolecular and intramolecular disulfide bonds. For example, an immobilized heterodimeric αβ T cell receptor protein complex possessing an intermolecular disulfide bond was used to compare relative noncovalent binding affinity to the complex of each of a series of peptide ligands [Garcia, K. C., et al. Proc Natl Acad Sci, USA (1997) 94, 13838-13843]. In another example, a disulfide bond was engineered into a C-terminal fragment of the B1 domain of protein G in order to examine binding of the C-terminal B1 domain fragment to the remaining N-terminal fragment of the protein G B1 domain, and effects of the disulfide bond on subsequent B1 domain stability [Kobayashi, N., et al. Biochemistry (1999) 38, 3228-3234]. In these examples, SPR was used to gain information about binding of a single ligand or protein folding. In contrast, the methods of the present invention relate to the use of SPR in the context of a plurality of substrates and/or binding partners and may be used in order to determine whether more than one binding partner can bind to the substrate simultaneously. Alternatively, the methods of the present invention relate to the use of SPR where a covalent bond is formed between an immobilized substrate and a binding partner, and may be used to rank relative affinity of a plurality of binding partners to a substrate.

### SUMMARY OF THE INVENTION

The present invention relates to the use of SPR methods to obtain information relating to molecular binding events. In one aspect of the present invention, methods are provided for determining whether a binding interaction is competitive or cooperative with respect to another binding interaction. In another aspect of the present invention, methods are provided for obtaining information where the binding interaction includes a formation of a reversible covalent bond.

### BRIEF DESCRIPTION OF THE FIGURES

FIG. 1 shows the BIACORE detection system.

FIG. 2 shows an illustrative example of an SPR experiment measuring the kinetics of association and the kinetics of dissociation.

FIG. 3 shows an illustrative binding experiment of three different compounds to IL-2. The compounds are each present at 250 μM.

FIG. 4 shows (A) competitive binding, (B) independent binding, and (C) cooperative binding of a first ligand and a second ligand (labeled with an asterisk) to an immobilized substrate-thiolate, where the second ligand is reversibly covalently attached by a disulfide bond to the immobilized substrate, thereby forming an immobilized substrate-SS—ligand complex. The first ligand is in solution and is allowed to flow over the immobilized complex. The first ligand is also allowed to flow over an immobilized substrate-thiolate corresponding to the substrate of the immobilized complex. In (A) the first ligand is unable to bind the immobilized substrate-SS—ligand complex, but is able to bind the corresponding substrate-thiolate. In (A)(i) the binding site of the second ligand overlaps the binding site of the first ligand on the substrate. In (A)(ii) the presence of the second ligand in the immobilized substrate-SS—ligand complex results in an allosteric conformational change, thereby removing the binding site of the first ligand. Mechanisms in (A)(i) and in (A)(ii) are not necessarily mutually exclusive. In (B), the first ligand is able to bind to a similar extent the immobilized substrate-SS—ligand complex and the corresponding immobilized substrate-thiolate. In (C) the first ligand binds the immobilized substrate-SS—ligand complex to a greater extent than the corresponding immobilized substrate-thiolate. The presence of the second ligand in the immobilized substrate-SS—ligand complex creates or improves a binding site for the first ligand.

FIG. 5 shows competitive binding between an immobilized ligand-thiolate and a ligand-thiolate in solution (labeled with an asterisk) to a substrate-thiolate having a masked thiol in solution. A substrate-thiolate is allowed to flow over the immobilized ligand-thiolate, thereby forming an immobilized substrate-SS—ligand complex having a reversible covalent bond, increasing the SPR signal and releasing RSH. Next a ligand-thiolate in solution is allowed to flow over the immobilized substrate-SS—ligand complex. Competitive binding by the ligand-thiolate in solution results in reversal of the covalent bond of the immobilized substrate-SS—ligand thiolate, the formation of a substrate-SS—ligand complex in solution, and a concomitant decrease of the SPR signal.

FIG. 6 shows the reactions for reversible covalent bond formation, using disulfide bond formation as a representative example. A ligand-thiolate flows over an immobilized substrate-thiolate having a masked thiol. Under appropriate conditions, interaction of the ligand-thiolate with the immobilized substrate-thiolate results in formation of a substrate-SS—ligand complex and release of RSH. The extent of the reversible bond formation can be monitored by SPR, because the immobilized substrate-SS—ligand complex and the immobilized substrate-thiolate have different masses. Measurement of the formation of the substrate-SS—ligand complex over time gives kinetics of the reversible covalent bond formation. Next, an agent that reverses the formation of the covalent bond, such as a chemical reductant, is added, and the dissociation of the substrate-SS—ligand complex to the immobilized substrate-thiolate and free ligand-thiolate is measured over time to give dissociation kinetics. The dissociation experiment may also be performed using increasing concentrations of the agent, where the extent of dissociation of the substrate-SS—ligand complex into the immobilized substrate-thiolate and ligand-thiolate in solution is measured at each concentration of the agent. The strength of the interaction between the immobilized substrate-thiolate and the ligand-thiolate is related to the concentration of agent necessary to disrupt the substrate-SS—ligand complex in a particular time frame.

FIG. 7 shows an overlay of sensorgrams of 1 flowing over wild-type IL-2. In the sensorgrams, time is plotted on the X-axis and refractive index units (raw RUs) are plotted on the Y-axis. The different traces correspond to several different concentrations (μM) of the compound. The injection of the compound at each concentration was followed by running buffer over the immobilized protein. A plateau in the refractive index is reached as the compound binds the protein, and the refractive index decreases with time as the compound dissociates from the protein.

FIG. 8 shows an overlay of sensorgrams of a constant concentration of 2c (50 μM) running over wild-type IL-2 in the presence of various concentrations (μM) of 1. These concentrations of 1 correspond to the different traces. The amount of IL-2 bound by 2c decreases in the presence of increasing concentrations of 1.

FIG. 9 shows an overlay of a dose-response binding curve (black circles) and a dose-response inhibition curve (white squares) measured for compounds that competitively bind wild-type IL-2. For the dose-response binding curve, the X-axis is the concentration of 1, and the Y-axis (right-hand side) is the fraction of IL-2 bound by 1 in the absence of 2c. For the dose-response inhibition curve, the Y-axis (left-hand side) is the fraction of IL-2 bound by 2c, in the presence of a particular concentration of 1 (shown on the X-axis).

FIG. 10 shows dose-response curves of IL-2 bound by 3c, in the presence of 50 μM of 2c (squares), in the presence of 50 μM of 16 (triangles), and in the absence of another compound (circles). The concentration of 3c is shown on the X-axis; on the Y-axis is shown the fraction of IL-2 bound to 3c.

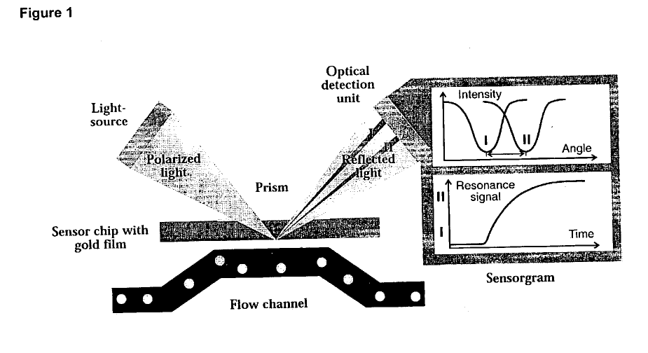

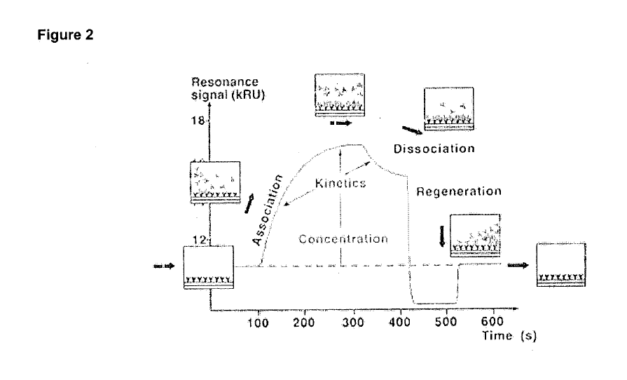

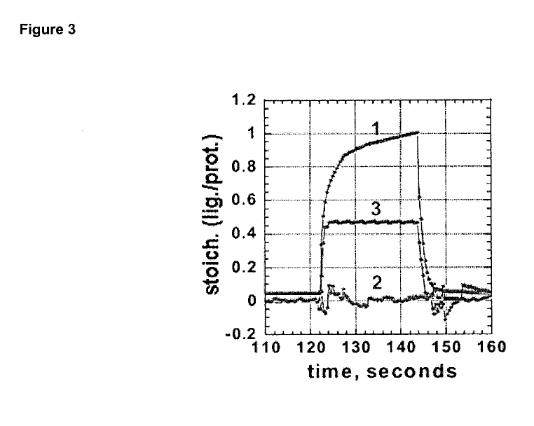

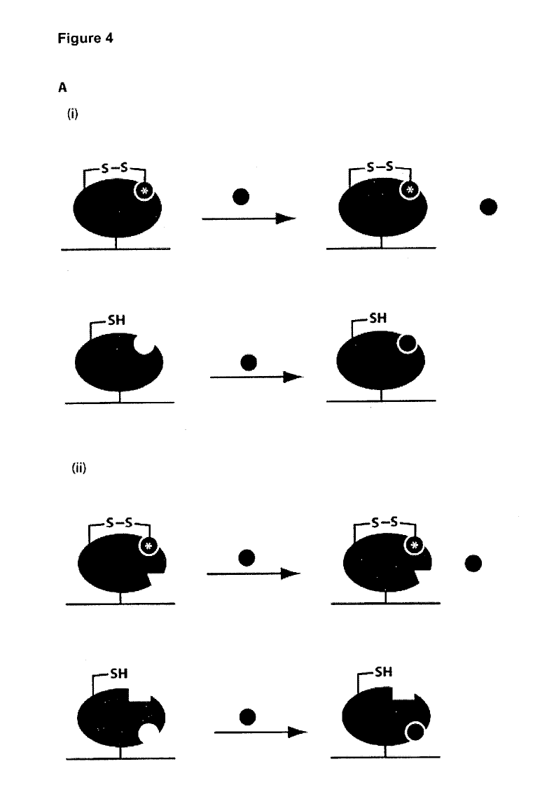

The binding of 3c by IL-2 increases in the presence of either 2c or 16.

### DETAILED DESCRIPTION OF THE PREFERRED EMBODIMENTS

Definitions

The definition of terms used herein include:

The term “aliphatic” or “unsubstituted aliphatic” refers to a straight, branched, cyclic, or polycyclic hydrocarbon and includes alkyl, alkenyl, alkynyl, cycloalkyl, cycloalkenyl, and cycloalkynyl moieties.

The term “alkyl” or “unsubstituted alkyl” refers to a saturated hydrocarbon.

The term “aryl” or “unsubstituted aryl” refers to mono or polycyclic unsaturated moieties having at least one aromatic ring. The term includes heteroaryls that include one or more heteroatoms within the at least one aromatic ring. Illustrative examples of aryl include: phenyl, naphthyl, tetrahydronaphthyl, indanyl, indenyl, pyridyl, pyrazinyl, pyrimidinyl, pyrrolyl, pyrazolyl, imidazolyl, thiazolyl, oxazolyl, isooxazoly, thiadiazolyl, oxadiazolyl, thiophenyl, furanyl, quinolinyl, isoquinolinyl, and the like.

The term “substituted” when used to modify a moiety refers to a substituted version of the moiety where at least one hydrogen atom is substituted with another group including but not limited to: aliphatic; aryl, alkylaryl, F, Cl, I, Br, —OH; —NO₂; —CN; —CF₃; —CH₂CF₃; —CH₂Cl; —CH₂OH; —CH₂CH₂OH; —CH₂NH₂; —CH₂SO₂CH₃; —OR^{x}; —C(O)R^{x}; —COOR^{x}; —C(O)N(R^{x})₂; —OC(O)R^{x}; —OCOOR^{x}; —OC(O)N(R^{x})₂; —N(R^{x})₂; —S(O)₂R^{x}; and —NR^{x}C(O)R^{x} where each occurrence of R^{x} is independently hydrogen, substituted aliphatic, unsubstituted aliphatic, substituted aryl, or unsubstituted aryl. Additionally, substitutions at adjacent groups on a moiety can together form a cyclic group.

The term “ligand” refers to an entity that possesses a measurable binding affinity for the target. In general, a ligand is said to have a measurable affinity if it binds to the target with a K_{d} or a Kᵢ of less than about 100 mM, preferably less than about 10 mM, and more preferably less than about 1 mM. In preferred embodiments, the ligand is not a peptide and is a small molecule. A ligand is a small molecule if it is less than about 2000 daltons in size, usually less than about 1500 daltons in size. In more preferred embodiments, the small molecule ligand is less than about 1000 daltons in size, usually less than about 750 daltons in size, and more usually less than about 500 daltons in size.

The phrase “reversible covalent bond” as used herein refers to a covalent bond that can be broken, preferably under conditions that do not denature the substrate. Examples include, without limitation, disulfides, Schiff-bases, thioesters, coordination complexes, boronate esters, and the like.

The phrase “reversing agent” as used herein is a chemical or biological molecule that is capable of reversing a reversible covalent bond. A nonlimiting example of a reversing agent is a chemical reductant that can disrupt a disulfide bond.

The phrase “chemically reactive group” is a chemical group or moiety providing a site at which a covalent bond can be made when presented with a compatible or complementary reactive group. Illustrative examples are —SH that can react with another —SH or —SS— to form a disulfide; an —NH₂ that can react with an activated —COOH to form an amide; an —NH₂ that can react with an aldehyde or ketone to form a Schiff base and the like.

The phrase “site of interest” refers to any site on a target on which a ligand can bind. For example, when the target is an enzyme, the site of interest can include amino acids that make contact with, or lie within about 10 Angstroms (more preferably within about 5 Angstroms) of a bound substrate, inhibitor, activator, cofactor, or allosteric modulator of the enzyme. When the enzyme is a protease, the site of interest includes the substrate binding channel from P6 to P6′, residues involved in catalytic function (e.g., the catalytic triad and oxy anion hole), and any cofactor (e.g., metal such as Zn) binding site. When the enzyme is a protein kinase, the site of interest includes the substrate-binding channel in addition to the ATP binding site. When the enzyme is a dehydrogenase, the site of interest includes the substrate binding region as well as the site occupied by NADINADH. When the enzyme is a hydrolase such as PDE4, the site of interest includes the residues in contact with cAMP as well as the residues involved in the binding of the catalytic divalent cations.

The term “substrate” is used the broadest sense, and refers to a chemical or biological entity for which the binding of a ligand has an effect on the function of the substrate. The substrate can be a molecule, a portion of a molecule, or an aggregate of molecules. The binding of a ligand may be reversible or irreversible. Specific examples of substrate molecules include polypeptides or proteins (e.g., enzymes, including proteases, e.g., cysteine, serine, and aspartyl proteases), receptors, transcription factors, ligands for receptors, growth factors, cytokines, immunoglobulins, nuclear proteins, signal transduction components (e.g., kinases, phosphatases), allosteric enzyme regulators, and the like, polynucleotides, peptides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. In a preferred embodiment, the substrate is a protein or a portion thereof or that comprises two or more amino acids, and which possesses or is capable of being modified to possess a reactive group that is capable of forming a covalent bond with a ligand having a complementary reactive group. The substrate can be obtained in a variety of ways, including isolation and purification from natural source, chemical synthesis, recombinant production and any combination of these and similar methods.

In one embodiment, the chemically reactive group on the substrate and the chemically reactive group each ligand are each independently a —SH or a masked —SH. An illustrative example of a masked thiol is a disulfide of the formula —SSR¹ where R¹ is as defined hereinafter. It is preferable that comparisons be made between ligand-thiolates having identical chemically reactive groups.

The present invention is directed to methods that use SPR to compare binding interactions between different ligands and a substrate. In one aspect, the ligands are presented simultaneously to the substrate for at least one step, and each interacts with the substrate solely through noncovalent interactions. In another aspect, binding of a plurality of ligands is measured sequentially or in parallel, and the interaction or binding between the ligands and the substrate involves the formation of a reversible covalent bond between the ligand and the substrate. In yet another aspect, the ligands are presented simultaneously for at least one step and at least one of the ligands is capable of forming a reversible covalent bond with the substrate.

In one embodiment, a method is provided for determining whether two ligands bind competitively, simultaneously, or cooperatively, wherein at least one of the ligands can form a reversible covalent bond with the substrate. The method comprises:

a) measuring by SPR a first extent of interaction between a first ligand and a substrate in the presence of a second ligand, wherein the substrate possesses a first chemically reactive group and the second ligand possesses a second chemically reactive group and the second ligand is capable of forming a reversibly covalent substrate-ligand complex with the substrate, and wherein the first ligand or the substrate is immobilized to a surface,

b) measuring by SPR a second extent of interaction between the first ligand and the substrate in the absence of the second ligand, and

c) comparing the first extent of interaction with the second extent of interaction.

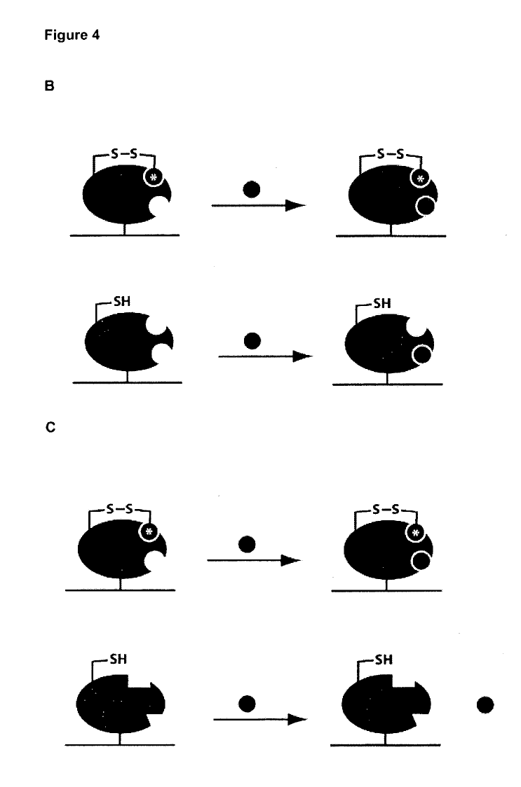

In one embodiment, the first ligand and the second ligand are binding cooperatively to the substrate and the first extent of interaction is greater than the second extent of interaction. In another embodiment, the first ligand and the second ligand are binding simultaneously to the substrate, and the first extent of interaction is equal to the second extent of interaction. In yet another embodiment, the first ligand and the second ligand are binding competitively to the substrate and the first extent of interaction is less than the second extent of interaction. Competitive binding, simultaneous binding, and cooperative binding are illustrated for one illustrative example of the method in FIG. 4(A), (B), and (C), respectively. As illustrated, the substrate is immobilized to the surface, and a first chemically reactive group is a first thiol group thereon. A second chemically reactive group is a second thiol group. A second ligand that does not include a thiol group (hereinafter referred to as the ligand) is then allowed to flow over the both sets of immobilized substrates and binding of the ligand to both surfaces is monitored by an increase in refractive index values. Reduced binding of the ligand to the substrate-SS—ligand complex indicates competition between the ligand and the ligand-thiolate (FIG. 4A). This competition may be due to overlapping binding sites, as illustrated in FIG. 4A(i). Alternatively, the binding of the ligand-thiolate causes an allosteric change in the substrate that is unfavorable to the binding of the ligand (FIG. 4A(ii)). Simultaneous binding occurs when the ligand binds equally to the substrate and the substrate-SS—ligand complex (FIG. 4B). In another embodiment, increased binding of the ligand to the substrate-SS—ligand complex relative to the substrate-thiolate indicates cooperative binding of the two ligands. In other words, the two compounds are capable of binding the ligand simultaneously, and the presence of one of the compounds facilitates the binding of the other. One mechanism for cooperative binding is illustrated in FIG. 4C, where the binding of the ligand-thiolate causes an allosteric change in the protein such that a binding site for the ligand is created or improved. Another mechanism for cooperative binding is where the binding of the ligand-thiolate forms at least part of a binding surface for the ligand.

In another embodiment of the method the first extent of binding and the second extent of binding are measured in parallel. For example, the substrate-SS—ligand complex and the substrate-thiolate are immobilized in different channels, and the first ligand is allowed to flow over both at the same time. In another embodiment, the measurements may be taken in sequence in the same channel. In one example, the first extent of binding is measured, and then a corresponding substrate that is a substrate-thiolate may be obtained from the substrate-SS—ligand complex by treatment with a chemical reductant followed by a wash step to remove the first and second ligands. Then the second extent of binding can be measured by readdition of the first ligand. In another example, the measurement of binding of the first ligand to a substrate-thiolate in step b) can be measured first, and the first ligand then washed off the chip. The substrate-thiolate can react on the chip to form the substrate-SS—ligand complex with the second ligand, the excess second ligand can be washed off, and the binding of the first ligand to the substrate-SS—ligand complex in step a) can then be measured.

One feature of the methods is the nature of the ligands and the substrate. In one embodiment, the substrate is a protein and at least one ligand is a small molecule ligand having a molecular weight <2000. In another embodiment, the substrate is a protein and at least one ligand is a soluble small molecule with a molecularweight <1500. In a preferred embodiment, the substrate is a protein and at least one ligand is a soluble small molecule with a molecular weight <1000. In another preferred embodiment, the substrate is a protein and at least one ligand is a soluble small molecule with a molecular weight <750. In another preferred embodiment, the substrate is a protein and at least one ligand is a soluble small molecule with a molecular weight <500. In another embodiment, the substrate is a protein and both ligands are soluble small molecules, each having a molecular weight <2000. In another embodiment, the substrate is a protein and both ligands are soluble small molecules each having a molecular weight <1000 Da. In another embodiment, the substrate is a protein and both ligands are soluble small molecules having a molecular weight <750.

The ligands may be identified by any number of screening methods, known to a skilled practitioner of the art. For example, the ligands may be identified by functional screening. The ligands may also be identified by the “tethering” method, which is described in U.S. Pat. No. 6,335,155, which issued on Jan. 1, 2002, and is incorporated here by reference. Alternatively, the ligands may be identified by SPR, and even by using methods of the present invention.

Another feature of methods described herein involving the presence or formation of a reversible covalent bond is the nature of the chemically reactive groups that react to form the reversible covalent bond. Synthetic methods for forming a reversible or irreversible covalent bond between reactive groups on a substrate and a ligand, are well known in the art, and are described in basic textbooks, such as, e.g., March, Advanced Organic Chemistry, John Wiley & Sons, New York, 4^{th} edition, 1992. Reductive aminations between aldehydes and ketones and amines are described, for example, in March et al., supra, at pp. 898-900; alternative methods for preparing amines at page 1276; reactions between aldehydes and ketones and hydrazide derivatives to give hydrazones and hydrazone derivatives such as semicarbazones at pp. 904-906; amide bond formation at p. 1275; formation of ureas at p. 1299; formation of thiocarbamates at p. 892; formation of carbamates at p. 1280; formation of sulfonamides at p. 1296; formation of thioethers at p. 1297; formation of disulfides at p. 1284; formation of ethers at p. 1285; formation of esters at p. 1281; additions to epoxides at p. 368; additions to aziridines at p. 368; formation of acetals and ketals at p. 1269; formation of carbonates at p. 392; formation of denamines at p. 1264; metathesis of alkenes at pp. 1146-1148 (see also Grubbs et al., Acc. Chem. Res. 28, 446-453 [1995]); transition metal-catalyzed couplings of aryl halides and sulfonates with alkanes and acetylenes, e.g. Heck reactions, at pp. 717-178; the reaction of aryl halides and sulfonates with organometallic reagents, such as organoboron, reagents, at p. 662 (see also Miyaura et al., Chem. Rev. 95, 2457 [1995]); organotin, and organozinc reagents, formation of oxazolidines (Ede et al., Tetrahedron Letts. 28, 7119-7122 [1997]); formation of thiazolidines (Patek et al., Tetrahedron Letts. 36, 2227-2230 [1995]); amines linked through amidine groups by coupling amines through imidoesters (Davies et al., Canadian J. Biochem. 50, 416-422 [1972]), and the like.

In one specific embodiment of the method, the substrate is a substrate-thiolate, the first chemically reactive group is a first thiol group, the second chemically reactive group is a second thiol group, and the second ligand is capable of forming a substrate-SS—ligand complex with the substrate-thiolate. In preferred embodiments, the substrate is a protein and the chemically reactive group is a thiol on a cysteine residue therein. If a site of interest does not include a naturally occurring cysteine residue, then the target can be modified to include a cysteine residue at or near the site of interest. A cysteine is said to be near the site of interest if it is located within 10 Angstroms from the site of interest, preferably within 5 Angstroms from the site of interest. Preferred residues for modification are those that are solvent-accessible. Solvent accessibility may be calculated from structural models using standard numeric (Lee, B. & Richards, F. M., J. Mol. Biol 55, 379-400 (1971); Shrake, A. & Rupley, J. A., J. Mol. Biol. 79, 351-371 (1973)) or analytical (Connolly, M. L. Science 221, 709-713 (1983); Richmond, T. J., J. Mol. Biol. 178, 63-89 (1984)) methods. For example, a potential cysteine variant is considered solvent-accessible if the combined surface area of the carbon-beta (CB), or sulfur-gamma (SG) is greater than 21 Å² when calculated by the method of Lee and Richards (Lee, B. & Richards, F. M. J. Mol. Biol 55, 79-400 (1971)). This value represents approximately 33% of the theoretical surface area accessible to a cysteine side-chain as described by Creamer et al. (Creamer, T. P. et al. Biochemistry 34, 6245-16250 (1995)).

It is also preferred that the residue to be mutated to cysteine, or another thiol-containing amino acid residue, not participate in hydrogen-bonding with backbone atoms or, that at most, it interacts with the backbone through only one hydrogen bond. Wild-type residues where the side-chain participates in multiple (>1) hydrogen bonds with other side-chains are also less preferred. Variants for which all standard rotamers (chi1 angle of −60⁰, 600, or 1800) can introduce unfavorable steric contacts with the N, CA, C, O, or CB atoms of any other residue are also less preferred. Unfavorable contacts are defined as interatomic distances that are less than 80% of the sum of the van der Waals radii of the participating atoms. In certain embodiments where the site of interest is a concave region, residues found at the edge of such a site (such as a ridge or an adjacent convex region) are more preferred for mutating into cysteine residues. Convexity and concavity can be calculated based on surface vectors (Duncan, B. S. & Olson, A. J., Biopolymers 33 219-229 (1993)) or by determining the accessibility of water probes placed along the molecular surface (Nicholls, A., et al., Proteins 11, 281-296 (1991); Brady, G. P., Jr. & Stouten, P. F., J. Comput. Aided Mol. Des. 14, 383-401 (2000)). Residues possessing a backbone conformation that is nominally forbidden for L-amino acids (Ramachandran, G. N., et al., J. Mol. Biol. 7 95-99 (1963); Ramachandran, G. N. & Sasisekharahn, V., Adv. Prot. Chem. 23, 283-437 (1968)) are less preferred targets for modification to a cysteine. Forbidden conformations commonly feature a positive value of the phi angle.

Other preferred variants are those which, when mutated to cysteine and reversibly covalently bound to a ligand or to a compound as to comprise —Cys—SSR¹, would possess a conformation that directs the atoms of R¹ towards the site of interest. Two general procedures can be used to identify these preferred variants. In the first procedure, a search is made of unique structures (Hobohm, U. et al. Protein Science 1,409-417 (1992)) in the Protein Databank (Berman, H. M. et al. Nucleic Acids Research 28, 235-242 (2000)) to identify structural fragments containing a disulfide-bonded cysteine at position j in which the backbone atoms of residues j−1, j, and j+1 of the fragment can be superimposed on the backbone atoms of residues i−1, i, and i+1 of the target molecule with an RMSD of less than 0.75 squared Angstroms. If fragments are identified that place the C β atom of the residue disulfide-bonded to the cysteine at position j closer to any atom of the site of interest than the C β atom of residue i (when mutated to cysteine), position i is considered preferred. In an alternative procedure, the residue at position i is computationally “mutated” to a cysteine and capped with an S-Methyl group via a disulfide bond.

In addition to adding one or more cysteines to a site of interest, it may be desirable to delete one or more naturally occurring cysteines (and replacing them with alanines for example) that are located outside of the site of interest. These mutants wherein one or more naturally occurring cysteines are deleted or “scrubbed” comprise another aspect of the present invention. Various recombinant, chemical, synthesis and/or other techniques can be employed to modify a target such that it possesses a desired number of free thiol groups that are available for tethering. Such techniques include, for example, site-directed mutagenesis of the nucleic acid sequence encoding the target polypeptide such that it encodes a polypeptide with a different number of cysteine residues. Particularly preferred is site-directed mutagenesis using polymerase chain reaction (PCR) amplification (see, for example, U.S. Pat. No. 4,683,195 issued Jul. 28, 1987; and Current Protocols In Molecular Biology, Chapter 15 (Ausubel et al., ed., 1991). Other site-directed mutagenesis techniques are also well known in the art and are described, for example, in the following publications: Ausubel et al., supra, Chapter 8; Molecular Cloning: A Laboratory Manual., 2nd edition (Sambrook et al., 1989); Zoller et al., Methods Enzymol. 100:468-500 (1983); Zoller & Smith, DNA 3 479-488 (1984); Zoller et al., Nucl. Acids Res., 10, 6487 (1987); Brake et al., Proc. Natl. Acad. Sci. USA 81, 4642-4646 (1984); Botstein et al., Science 229,1193 (1985); Kunkel et al., Methods Enzymol. 154, 367-82 (1987), Adelman et al., DNA 2 183 (1983); and Carter et al., Nucl. Acids Res., 13, 4331 (1986). Cassette mutagenesis (Wells et al., Gene, 34, 315 [1985]), and restriction selection mutagenesis (Wells et al., Philos. Trans. R. Soc. London SerA, 317, 415[1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g., separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

In one embodiment, the substrate possesses a thiol group as the first chemically reactive group (the substrate-thiolate). In another embodiment, the substrate-thiolate possesses a naturally occurring —SH group from a cysteine that is part of the naturally occurring protein sequence. In another embodiment, the substrate-thiolate possesses an engineered —SH group where mutagenesis was used to mutate a naturally occurring amino acid to a cysteine.

In another embodiment, the thiol group on the substrate-thiolate is masked as a disulfide. In another embodiment, thiol on the substrate is from a cysteine where the thiol is masked as a disulfide. In another embodiment, the substrate-thiolate forms a disulfide bond with another cysteine. In another embodiment, the substrate-thiolate forms a disulfide bond with glutathione. In another embodiment, the substrate-thiolate forms a disulfide of the formula —SSR¹ where R¹ is unsubstituted C₁-C₁₀ aliphatic, substituted C₁-C₁₀ aliphatic unsubstituted aryl or substituted aryl. In another embodiment, the substrate-thiolate forms a disulfide of the formula —SSR²R³ wherein R² is C₁-C₅ alkyl and R³ is NH₂, OH, or COOH. In another embodiment, the substrate-thiolate forms a disulfide of the formula —SSCH₂CH₂OH. In yet another embodiment, the substrate-thiolate possesses a cysteine where the thiol is masked as a disulfide of the formula —SSCH₂CH₂NH₂.

In another embodiment, the second ligand possesses a thiol group as the second chemically reactive group and is referred to as the ligand-thiolate. The ligand-thiolate is a ligand possessing a thiol group or an equivalent group that can be reacted with a thiolate to form a disulfide bond. In one embodiment, the ligand-thiolate possesses an —SH group. In another embodiment, the ligand-thiolate possesses a masked thiol. In another embodiment, the ligand-thiolate possesses a masked thiol in the form of a disulfide of the formula —SSR¹ where R¹ is unsubstituted C₁-C₁₀ aliphatic, substituted C₁-C₁₀ aliphatic, unsubstituted aryl or substituted aryl. In another embodiment, the ligand-thiolate possesses a thiol masked as a disulfide of the formula —SSR²R³ wherein R² is C₁-C₅ alkyl (preferably —CH₂—, —CH₂CH₂—, or —CH₂CH₂CH₂—) and R³ is NH₂, OH, or COOH. In another embodiment, the ligand-thiolate possesses a thiol masked as a disulfide of the formula —SSCH₂CH₂OH. In yet another embodiment, the ligand-thiolate possesses a thiol masked as a disulfide of the formula —SSCH₂CH₂NH₂.

In a preferred embodiment, the substrate has a first thiol group as the first chemically reactive group, herein referred to as the substrate-thiolate, and the substrate-thiolate is protein mutated to possess the thiol, for example, a mutation of a noncysteine wild-type residue to a cysteine. The substrate-thiolate and the corresponding substrate-SS—ligand complex are used in the methods described herein. However, under certain circumstances, a version of the substrate-thiolate without the thiol can be used when the binding of a ligand of interest is equivalent to both the substrate-thiolate and the version of the substrate without the thiol.

Another feature of the invention is where the ligand and not the substrate is immobilized to the surface. Example 3 gives a representative protocol for this method and Example 4 shows results representative data for compounds binding to interleukin-2.

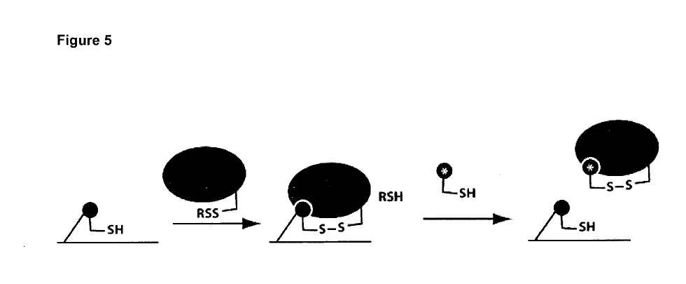

In another embodiment, the first ligand is immobilized to the surface and possesses a thiol group. FIG. 5 shows a representative example of this embodiment. As shown, the first ligand is a ligand-thiolate that is immobilized to the surface, and the substrate is allowed to flow over, and an extent of binding is measured in the absence of the second ligand. In the figure, the immobilized ligand has an —SH group, and the substrate-thiolate is masked with an —SR group. Suitable R groups are as described herein. Alternatively, the immobilized ligand can possess the masking group and the substrate-thiolate can possess the —SH group. Next, the binding of the first ligand to the substrate-thiolate is measured in the presence of a second ligand-thiolate that is also capable of forming a reversible covalent bond with the substrate, thereby displacing the substrate from the first ligand. In this case, a decrease in the refractive index would indicate competitive binding of the first ligand and the second ligand-thiolate for the substrate.

In another aspect of the invention a method is provided for measuring binding of ligands to a substrate, specifically where at least one of the ligands is capable of forming a disulfide bond with the substrate. The method comprises:

a) measuring by SPR a first extent of interaction between a first ligand and a substrate-thiolate in the presence of a second ligand, wherein the substrate-thiolate possesses a first thiol group, and the second ligand possesses a second thiol group, and the substrate-thiolate and the second ligand are capable of forming a substrate-SS—ligand complex, and wherein the first ligand or the substrate-thiolate is immobilized to a surface,

b) measuring by SPR a second extent of interaction between the first ligand and the substrate-thiolate in the absence of the second ligand, and

c) comparing the first extent of interaction with the second extent of interaction.

In one embodiment of the method, the following conditions apply:

(i) the first extent of binding and the second extent of binding are measured where the first ligand is immobilized to the surface, and

(ii) the first extent of binding is measured under conditions where the substrate-thiolate and the second ligand have formed a free substrate-SS—ligand complex.

In still another embodiment of the method, the following conditions apply:

(i) the first ligand is a ligand-thiolate and is immobilized to the surface,

(ii) the first extent of binding is the extent of formation of an immobilized ligand-SS—substrate complex between the immobilized ligand-thiolate and the substrate-thiolate having the first thiol group in the presence of the second ligand having the second thiol group, and

(iii) the second extent of binding indicates the extent of formation of the immobilized ligand-SS—substrate complex in the absence of the second ligand.

In yet another embodiment of the method the following conditions apply:

(i) the substrate-thiolate is immobilized to the surface, and

(ii) the first extent of binding is measured where the substrate-thiolate and the second ligand have formed an immobilized substrate-SS—ligand complex.

In another aspect of the present invention, methods are provided for comparing binding interactions of a substrate and a ligand in a plurality of ligands, where the binding event includes formation of a reversible covalent bond between a first chemically reactive group on the substrate and a second chemically reactive group on the ligand. The interactions of each of the ligands to the substrate may subsequently be compared or ranked to relative to interactions of other ligands to the substrate. Alternatively, the interactions of an individual ligand to different sites of interest on the same substrate may be compared. Illustrative examples of reversible covalent bonds include disulfides, oximes, imines and the like. In preferred embodiments, the reversible covalent bond is a disulfide bond. Illustrative examples of such a system include a substrate (such as a protein or DNA), which either includes or has been modified to include a first thiol group and a ligand containing a second thiol group. Example 5 contains a protocol for measuring binding of a substrate and a ligand where the binding involves the formation of a reversible covalent bond, and Example 6 shows comparative results for different ligands binding at two sites of interest on the interleukin-1 receptor. Pertaining to this aspect of the invention, a method is provided to rank the relative interaction strength of ligands to a site of interest on substrate by using SPR to determine their relative abilities to form the reversible covalent bond in the presence of an agent that can reverse the reversible covalent bond (reversing agent). The method comprises:

a) measuring by SPR a calibration corresponding to an extent of formation of a reversible covalent bond between a substrate and a ligand in the presence of a reversing agent at a first concentration wherein the first concentration is sufficient to disrupt the reversible covalent bond and wherein the substrate or the ligand is immobilized,

b) measuring by SPR a plurality of test values wherein each value corresponds to an extent of formation of the reversible covalent bond between the substrate and the ligand at a concentration of a reversing agent that is less than the first concentration, and

c) correlating the extent of formation of the reversible covalent bond as a function of the concentration of the reversing agent.

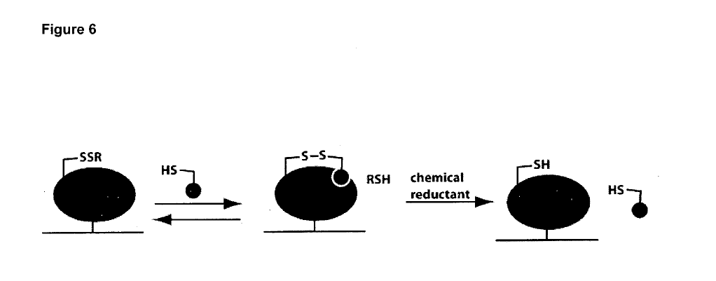

In a preferred embodiment, the first chemically reactive group is a first thiol group, the second chemically reactive group is a second thiol group, the reversible covalent bond is a disulfide bond, and the reversing agent is a chemical reductant. A substrate that either includes or has been modified to include a first chemically reactive group (substrate-thiolate) is immobilized to the surface and a ligand containing a second chemically reactive group (ligand-thiolate) is allowed to flow over the substrate surface (FIG. 6). The figure shows the substrate-thiolate as having a masked thiol, and the ligand thiolate as having an —SH group. Alternatively, the substrate may have the —SH group and the ligand may have the masked thiol. The extent of binding, accompanied by disulfide bond formation between the substrate-thiolate and the ligand-thiolate is noted by an increase in refractive index and is dependent on the concentration of ligand-thiolate, the affinity of the ligand-thiolate for the substrate-thiolate, and the concentration of reductant (the reduction potential) of the medium. Example 7 contains a representative protocol for measuring equilibrium binding using varying concentrations of a chemical reductant. Illustrative examples of suitable chemical reductant include but are not limited to: cysteine, cysteamine, dithiothreitol, dithioerythritol, glutathione, 2-mercaptoethanol, 3-mercaptoproprionic acid, a phosphine such as tris-(2-carboxyethyl-phosphine) (“TCEP”), or sodium borohydride. In one embodiment, the chemical reductant is 2-mercaptoethanol. In another embodiment, the chemical reductant is cysteamine. In another embodiment, the chemical reductant is glutathione. In another embodiment, the chemical reductant is cysteine. Example 8 contains a representative protocol for the measurement of disruption of a substrate-SS—ligand complex.

In another embodiment, the substrate is a protein and the ligand is a small molecule ligand having a molecular weight <2000. In another embodiment, the substrate is a protein and the ligand is a soluble small molecule with a molecular weight <1500. In another embodiment, the substrate is a protein and the ligand is a soluble small molecule with a molecular weight <1000. In another embodiment, the substrate is a protein and the ligand is a soluble small molecule with a molecular weight <750. In another embodiment, the substrate is a protein, and the ligand is a soluble small molecule with a molecular weight <500.

In another aspect of the present invention, a method is provided for determining whether two ligands bind to a substrate competitively or cooperatively, wherein the ligands need not have a reversibly covalent interaction with the substrate. In particular, one embodiment is a method of determining whether a first ligand and a second ligand bind competitively or cooperatively to a substrate. The method comprises:

a) measuring a first SPR value of an immobilized binding partner in the presence of a nonimmobilized binding partner in a solution, wherein one binding partner is a substrate and the other binding partner is the first ligand,

b) adding the second ligand to the solution,

c) measuring a second SPR value that results, and

d) comparing the first SPR value with the second SPR value;

wherein at least one ligand is a small molecule ligand;

wherein the first ligand and the second ligand do not necessarily interact with one another in the absence of the substrate; and

wherein if the mass of the first ligand is greater than the mass of the second ligand, and the first ligand and the second ligand each bind the substrate with a 1:1 stoichiometry, then a decrease in the second SPR value indicates competitive binding of the first ligand and the second ligand to the substrate, and an increase in the second SPR value indicates potential simultaneous and potential cooperative binding of the first ligand and the second ligand to the substrate. In a particular embodiment, the substrate is a protein. In another embodiment, the substrate is a protein and at least one ligand is a small molecule ligand having a molecular weight <2000. In yet another embodiment the immobilized binding partner is the substrate. In a preferred embodiment, the immobilized binding partner is the substrate, and the first ligand is present in a saturating amount. In another embodiment, the immobilized binding partner is the first ligand. In another embodiment, the immobilized binding partner is the first ligand, and the substrate is present in a subsaturating amount.

In one embodiment, the substrate is immobilized onto the surface, and the first ligand is not immobilized, and both ligands bind to the substrate solely through noncovalent interactions. A representative protocol for this embodiment is given in Example 9. In general, the ligand having the higher molecular weight is allowed to flow over the immobilized substrate at a constant concentration and the extent of binding is measured by the change in refractive index relative to buffer. Varying concentrations of a ligand having a lower molecular weight are then allowed to flow over the substrate in the presence of a constant concentration of the ligand with the higher molecular weight and the changes in refractive index are noted. If a reduction in the refractive index is observed upon the addition of ligand having the lower molecular weight, then the ligand having lower molecular weight is a competitor of the ligand of higher molecular weight.

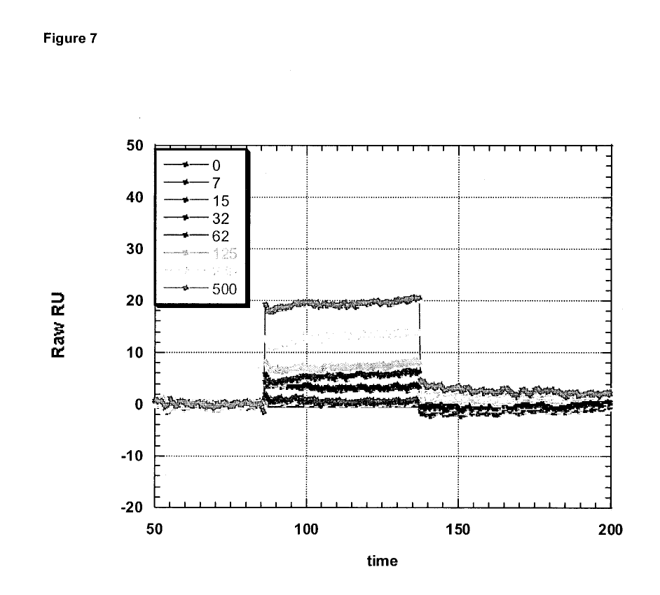

FIG. 7 shows an overlay of sensorgrams obtained by adding a single ligand (1) at different concentrations to a substrate. The plateau regions in the RU values show binding by the ligand; the higher the plateau, the greater the extent of binding.

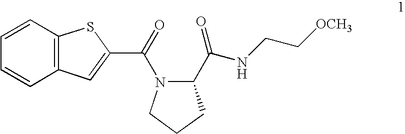

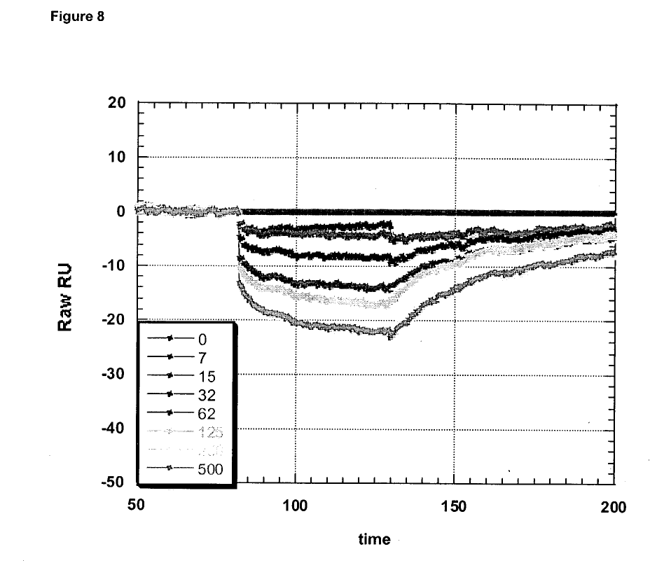

FIG. 8 shows a corresponding overlay of sensorgrams where 2c is the ligand of higher molecular weight that is held at constant concentration and 1 is the ligand of lower molecular weight at variable concentrations. A reduction in refractive index is observed upon increasing concentrations of 1 added to 2c.

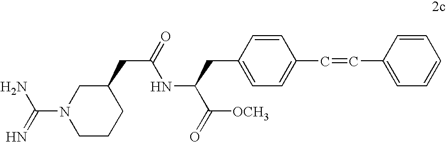

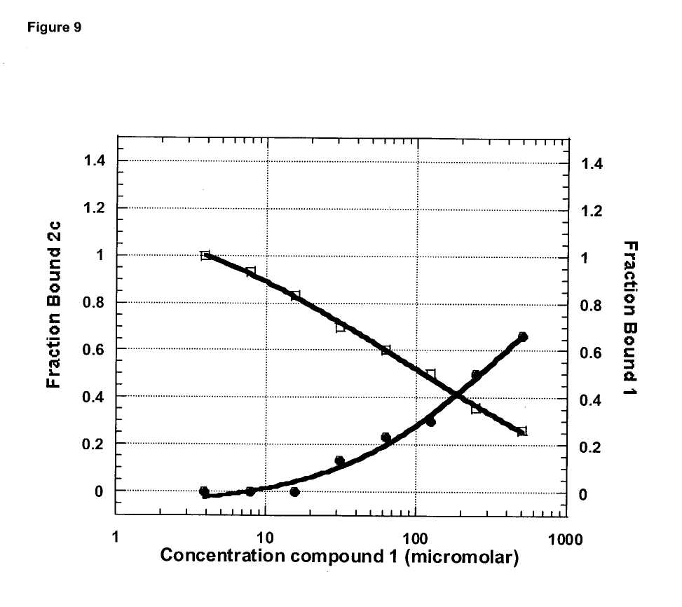

FIG. 9 shows the corresponding dose-response plots. As can be seen, 1 causes 2c to be removed from the substrate. The ability of the ligand having lower molecular weight to compete with the ligand of higher molecular weight indicates that there may be overlapping binding sites. Alternatively, the binding of 1 causes an allosteric change in the substrate that is unfavorable to the binding of 2c. Example 10 shows representative data obtained from use of the method; noncovalently binding ligands were found by SPR to bind a substrate in a competitive fashion.

In another embodiment of the method wherein the immobilized binding partner is the substrate and the nonimmobilized binding partner is the first ligand, the method further comprises:

a) measuring a third SPR value of the immobilized substrate in the absence of a ligand in the same solution or in a parallel solution,

b) adding the second ligand to the solution in step e),

c) measuring a fourth SPR value that results, and

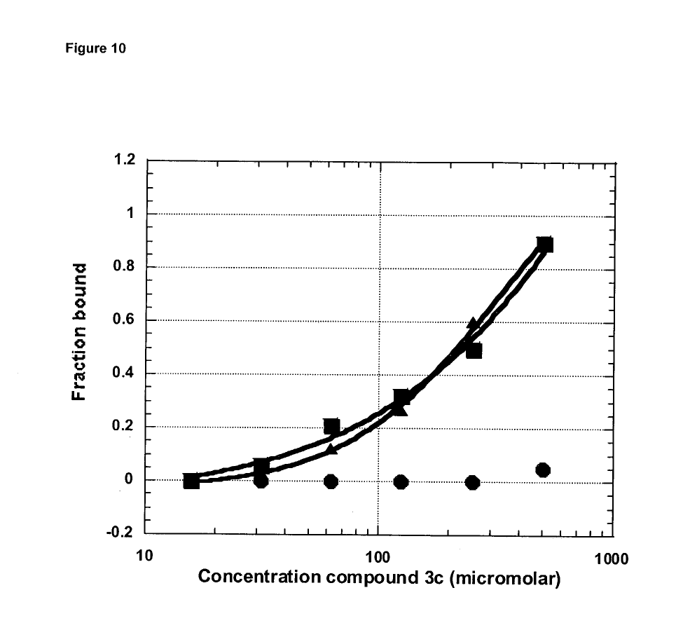

d) comparing the third SPR value with the fourth SPR value;

where a greater increase between the first and the second SPR values compared to an increase between the third and fourth SPR values for equal amounts of second ligand added to produce the second SPR value and the fourth SPR value indicates cooperative binding of the first ligand and the second ligand to the immobilized substrate. In another words, both the first and second ligands are binding simultaneously to the substrate and the binding of one facilitates the binding of the other. The representative protocol given in Example 9 was used to determine cooperative binding between two ligands in solution to an immobilized substrate. The dose response curve of the second ligand is measured in the presence and absence of the first ligand; a representative set of dose-response curves are shown in FIG. 10. The differences in the measured affinity in these two situations are indicative of the degree of cooperative binding. Example 11 provides representative results for ligands binding cooperatively to an immobilized substrate.

In another embodiment, a method is provided for assessing the ability of two ligands to compete against each other for binding to a substrate. In this embodiment, the first ligand is immobilized and the substrate is nonimmobilized. A second ligand is then added to the protein solution. If reduction in the refractive index is observed relative to the substrate only measurement, then competitive binding for ligand 1 and ligand 2 is established. Conversely, if an increase in refractive index is observed relative to the substrate only measurement, potential cooperative binding of ligand 1 and ligand 2 is indicated. In particular, the method for determining whether two molecules bind competitively or cooperatively to a substrate comprises:

a) measuring a first SPR value of an immobilized binding partner in the presence of a nonimmobilized binding partner in a solution, wherein one binding partner is a substrate and the other binding partner is the first ligand,

b) adding the second ligand to the solution,

c) measuring a second SPR value that results, and

d) comparing the first SPR value with the second SPR value;

wherein at least one ligand is a small molecule ligand;

wherein the first ligand and the second ligand each bind the substrate with a 1:1 stoichiometry;

wherein the first ligand and the second ligand do not necessarily interact with one another in the absence of the substrate; and

wherein if the mass of the first ligand is greater than the mass of the second ligand, then a decrease in the second SPR value indicates competitive binding of the first ligand and the second ligand to the substrate, and an increase in the second SPR value indicates potential simultaneous and potential cooperative binding of the first ligand and the second ligand to the substrate;

where the immobilized binding partner is the first ligand and the nonimmobilized binding partner is the substrate. In yet another embodiment, the method further comprises:

e) measuring a third SPR value of the immobilized ligand in the absence of a substrate in the same solution or in a parallel solution;

f) adding the substrate to the solution in step e); and

g) measuring a fourth SPR value that results;

where a greater increase between the first and the second SPR values compared to an increase between the third and fourth SPR values for equal amounts of the substrate added to produce the second SPR value and the fourth SPR value indicates cooperative binding of the first ligand and the second ligand to the nonimmobilized substrate. In one embodiment of the method, the first ligand, the substrate, and the second ligand interact with one another solely through noncovalent interactions. Example 12 gives a representative protocol, and Example 13 provides results from use of the method to determine that an immobilized ligand and a ligand in solution engage in cooperative binding to a substrate in solution.

### EXAMPLE 1

Protocol for Ligand Binding to an Immobilized Protein-SS—Ligand Complex

A CM5 sensor chip (Biacore-BR-1000-14) is docked in a Biacore 2000 SPR device, and the chip is then normalized using a standard Biacore protocol with 70% glycerol. The chip is next equilibrated in PBS for approximately 1 hr at a 20 μL/min flow rate. The dextran surface of the chip is activated through an NHS/EDC coupling. This activation is done using an amine coupling kit (Biacore—BR-100-50) containing N-hydroxysuccinimide (NHS 11.5 mg/mL) and N-ethyl-N′-(3-dimethylaminopropyl) carbodiimide hydrochloride (EDC 75 mg/mL). The two reagents are first mixed in a 1:1 ratio and then 35 μL of the mixture are injected at 5 mL/min. The chip is activated one flow cell at a time, and that flow cell is subsequently labeled with protein.

IL-2 cysteine mutants are expressed in E. coli cells and purified using standard procedures. The cysteine mutants are treated with 10 mM βME overnight to reduce the surface cysteine. The βME is removed and the protein-thiolate is placed in 100 mM HEPES buffer pH 7.4 (Nap-5 column, Pierce). Compounds that have been shown previously to bind IL-2 cysteine mutants and participate in specific disulfide exchange reactions were chosen. The deblocked protein-thiolate is then treated with 100 μM compound and 100 μM βME for 2 hr. The free compound and βME are then removed and the protein-SS—ligand complex is placed in 100 mM MES buffer pH 5.75 (Nap-5 column, Pierce).

The protein-SS—ligand complex is next concentrated to 100 μM using a Millipore-UFV5BCCOO 5K NMWL membrane, 0.5 mL centrifugal filter and tube. A sensorgram is then run over the activated flow cell at 2 μL/min of PBS. The IL-2-SS—ligand solution is then injected at 2 μL/min for 10 min. After injection the change in RUs is measured by marking the RUs at t=0 and t=final and subtracting. This difference gives the total RU change that is related to the total mass of protein-SS—ligand successfully coupled to the CM5 surface. This value will later be used to determine stoichiometry of binding. The procedure is then repeated with wild type IL-2, which is immobilized on an adjacent cell. Proteins and protein-SS—ligand complexes are immobilized at densities of 100-160 fmol/mm². The sensorgram is then altered to flow across all four cells on the CM5 chip at 5 μL/min PBS. A solution of 1 M ethanolamine hydrochloride-NaOH pH 8.5 (Amine coupling kit, Biacore—BR-100-50) is injected at 5 μL/min for 7 min, and the chip is equilibrated overnight in PBS+0.05% azide+1% DMSO at 20 μL/min.

Dilution series of the compounds are typically performed to produce seven solutions with compound concentrations of 50 mM, 25 mM, 12.5 mM, 6.2 mM, 3.1 mM, 1.6 mM, 0.78 mM in DMSO, and a final eighth solution is a DMSO-only baseline. The compounds are then diluted into PBS+0.05% azide to create a second set of solutions having respective final concentrations of 500 μM, 250 μM, 125 μM, 62 μM, 31 μM, 16 μM, and 7.8 μM. The compound solutions are injected from lowest concentration to highest at 40 μL/min for 1 min. The SPR software method includes a 1 min wash before injection, the injection and then a 3 min wash after the injection.

The dilution series are analyzed by BIAevaluation software version 3.0. Sensorgrams are plotted for compound binding to the cells of the chip labeled with wt IL-2, and cells of the chip labeled with IL-2—SS— ligand. The sensorgrams are modified by subtracting the sensorgrams for the appropriate compound dilutions over cell 1 (a blank cell). This subtraction step removes any RU changes that might occur due to non-specific interactions between the compound and the chip itself or refractive changes in the buffer. Finally, the eight sensorgrams produced from the compound dilution series are zeroed at the average of their starting RUs. Then the sensorgram produced by injecting DMSO only is subtracted to reveal the final sensorgrams used to determine relative binding.

To determine K_{dSPR} for the compound, the compound concentration is plotted on the X-axis, while the fraction bound at the plateau of the injection is plotted on the Y-axis. The curve is then analyzed via non-linear regression analysis (Kaleidagraph, Synergy Software).

### EXAMPLE 2

Cooperative and Competitive Binding of Ligands to Immobilized Interleukin-2

The substrate-SS—ligand complex IL-2-SS—2 was made by creating a K43C mutant of human IL-2, and labeling the mutant protein with 2t, which is 2c modified to possess a thiol group. Specifically, the methoxy group was replaced by a —NHCH₂CH₂SSCH₂CH₂NH₂ group, which is a disulfide linker designed to form a disulfide with the substrate cysteine thiol, via removal of —SCH₂CH₂NH₂. Also shown below are the structures of 3c, 4c, and their respective analogs, 3t and 4t.

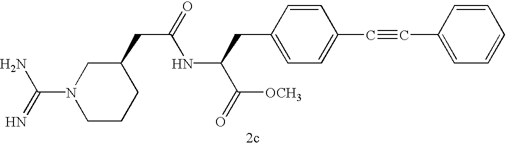

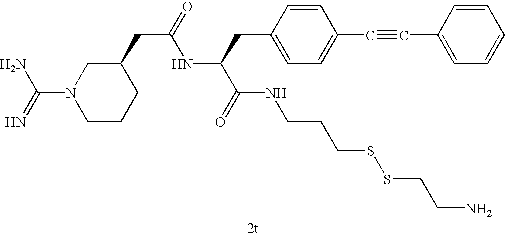

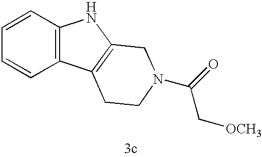

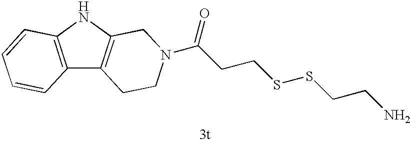

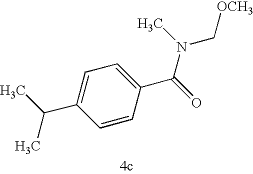

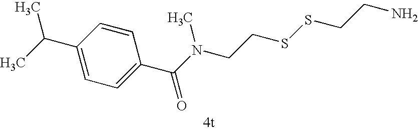

IL-2-SS—2 was immobilized in one channel, wild type human IL-2 was immobilized in a different channel, and the relative ability of compounds 3c and 4c to bind the two IL-2 species was measured by SPR. The reciprocal experiment was also performed by measuring relative binding of 2c to wild-type IL-2 and Y31 C-IL-2-SS—3 or Y31 C-IL-2-SS—4. Results are shown in Table 1.

TABLE 1

| | IL-2 wt K_{d} | Substrate-SS -ligand | IL-2-SS -ligand K_{d} |
|---|---|---|---|
| Compound | (μM) | complex | (μM) |
| 2c | 33 | Y31C-IL-2-SS -3 | 8 |
| 2c | 33 | Y31C-IL-2-SS -4 | 30 |
| 3c | Inactive | K43C-IL-2-SS -2 | 250 |
| 4c | Inactive | K43C-IL-2-SS -2 | 210 |

As Table 1 shows, neither 3c nor 4c binds to wild-type IL-2. However, both show affinity in the 200 μM range for the IL-2-SS—2 conjugate. This shows that the binding of these compounds to IL-2 is cooperative with the binding of 2. Compound 2c additionally shows increased affinity for the Y31-IL-2-SS—3 conjugate, as compared with the wild-type protein, and similar affinity for the Y31-1L-2-SS—4 conjugate, as compared with the wild-type protein.

### EXAMPLE 3

Protocol for Binding of a Protein-SS—Ligand Complex to an Immobilized Ligand

A CM5 sensor chip (Biacore-BR-1000-14) is docked in a Biacore 2000 SPR device, and the chip is normalized using a standard Biacore protocol with 70% glycerol. The chip is then equilibrated in PBS for approximately 1 hr at 20 μL/min flow rate. The dextran surface of the chip is activated through an NHS/EDC coupling. This activation is done using an amine coupling kit (Biacore—BR-100-50) containing N-hydroxysuccinimide (NHS 11.5 mg/mL) and N-ethyl-N′-(3-dimethylaminopropyl) carbodiimide hydrochloride (EDC 75 mg/mL). The two reagents are mixed in a 1:1 ratio and then 35 μL of the mixture are injected at 5 μL/min.

A ligand that is a micromolar inhibitor for IL-2 (2c, for structure see Example 2) was modified so as to contain reactive amine off a handle in the center of the molecule. The resulting molecule (2a) has the —OCH₃ group of 2c replaced with an —NCH₂CH₂OCH₂CH₂NH₂ group. The molecule is attached to the surface by reaction to form an amide bond between the terminal —NH₂ of the —NCH₂CH₂OCH₂CH₂NH₂ group and a surface carboxylate group. To immobilize the ligand 2a a 1:10 mixture of 2a and benzylamine was made with a final 2a concentration of 100 μM in 100 mM HEPES buffer pH 7.4. The small amine is used as filler so that the immobilized small ligand is not saturating the chip, but is instead spaced at intervals on the chip's surface. This solution containing the ligand and the filler is then injected over the activated cell at 5 μL/min for 4 min. After injection the change in RUs is measured by marking the RUs at t=0 and t=final and subtracting. This gives the total RU change that is related to the total mass of ligand successfully coupled to the CM5 surface. The compound is immobilized with a density of 8 fmol/mm². The sensorgram is then altered to flow across all four cells on the CM5 chip at 5 μL/min PBS. A solution of 1 M ethanolamine hydrochloride-NaOH pH 8.5 (Amine coupling kit, Biacore—BR-100-50) is injected at 5 μL/min for 7 min. The chip is equilibrated overnight in PBS+0.05% azide at 20 μL/min.

IL-2 cysteine mutants are expressed in E. coli cells and purified using standard procedures. The cysteine mutants are treated with 10 mM βME overnight to reduce the surface cysteine. The βME is removed and the protein is placed in 100 mM HEPES buffer pH 7.4 (Nap-5 column, Pierce). Compounds that have been shown previously to bind IL-2 cysteine mutants and participate in specific disulfide exchange reactions are chosen. The deblocked protein is then treated with 100 μM compound and 100 μM βME for 2 hr at room temperature. The free compound and βME is removed and the protein-SS—ligand complex is placed in PBS+0.05% azide (Nap-5 column, Pierce).

Dilution series of the proteins in PBS+0.05% azide are typically performed such that there are seven solutions with final protein concentrations of 7000 nM, 3500 nM, 1750 nM, 875 nM, 438 nM, 219 nM, and 109 nM; a final eighth point is used as a PBS-only baseline. The protein solutions, i.e., the IL-2 solution and the IL-2—SS— ligand solution, are each injected from lowest concentration to highest at 40 μL/min for 1 min. The SPR software method includes a 1 min wash before injection, the injection and then a 3 min wash after the injection.

These dilution series are analyzed by BIAevaluation software version 3.0. Sensorgrams are plotted for protein binding to the compound labeled cell of the chip. The sensorgrams are modified by subtracting the sensorgrams for the appropriate protein dilutions over cell 1 (a blank cell). This step removes any RU changes that might occur due to nonspecific interactions between the protein or the protein-SS—ligand complex and the chip itself, or due to refractive changes in the buffer. Finally, the eight sensorgrams produced from each protein dilution series are zeroed at the average of their starting RUs. Then the sensorgram produced by injecting PBS only is subtracted to reveal the final sensorgrams used to determine relative binding.

To determine K_{dSPR} for the protein (or the protein-SS—ligand complex), the protein (or the protein-SS—ligand complex) concentration is plotted on the X-axis, while the fraction bound at the plateau of the injection is plotted on the Y-axis. The curve is then analyzed via nonlinear regression analysis (Kaleidagraph, Synergy Software).

### EXAMPLE 4

Competitive and Cooperative Binding of Ligands to Nonimmobilized Interleukin-2

In these experiments, 2a was immobilized, and various protein-SS—ligand conjugates were flowed over 2a. Binding of the conjugates to 2a was compared to wild type IL-2 binding to 2a. The structures of the compounds used to create the IL2-SS—ligand conjugates are given in Table 2. As in Example 2, the —SCH₂CH₂NH₂ group is removed, and the remainder of the compound forms a disulfide bond with a cysteine group that has been introduced onto IL-2.

TABLE 2

| Compound | Structure |
|---|---|
| 5t | 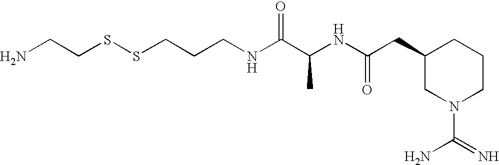 |
| 6t | 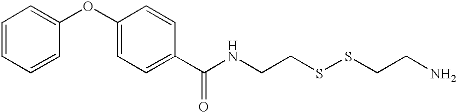 |
| 7t | 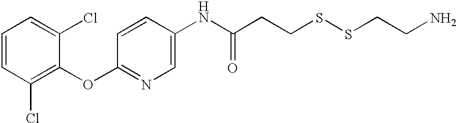 |
| 8t | 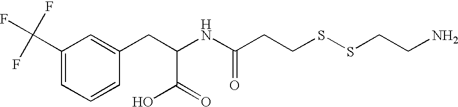 |
| 9t | 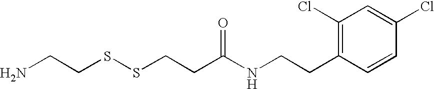 |
| 10t | 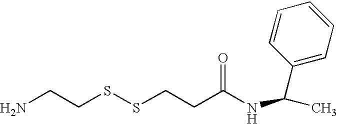 |
| 11t | 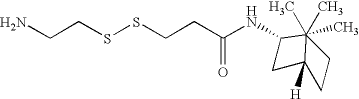 |

The binding results to wild-type substrate and to various substrate-SS—ligand complexes of immobilized 2a are shown in Table 3 below. It can be seen that immobilized 2a binds to wild-type IL-2 with an affinity of approximately 4 μM. Some of the substrate-SS—ligand complexes demonstrate an affinity for the immobilized ligand that is equal to that of the wild type protein for the immobilized ligand. Other substrate-SS—ligand conjugates show an increased affinity compared to that of the substrate for 2a. For example, the covalent complex Y31 C-IL-2-4 shows greater affinity for 2a than does the substrate, wild-type IL-2; the increase in affinity indicates that 4 and 2a are binding to the substrate in a cooperative manner. Still other substrate-SS—ligand complexes show a weaker affinity for 2a than does the wild type substrate IL-2. An example of a substrate-SS—ligand complex showing decreased affinity compared with the wild-type substrate for 2a is K43C-IL-2-5; the decrease in affinity is an indication that 2a and 5 are binding to IL-2 in a competitive manner.

TABLE 3

| Substrate or substrate- | Binding compared to wild | |
|---|---|---|
| SS -ligand | type substrate | K_{d,SPR} (1), nM |
| Wt IL-2 | — | 4000 |
| Y31C-IL-2 -4 | Augmented | 950 |
| K43C-IL-2 -5 | Inhibited | >7000 |
| L72C-IL-2 -6 | Inhibited | >7000 |
| Y31C-IL-2 -7 | Equal | 3500 |
| Y31C-IL-2 -8 | Equal | 3700 |
| Y31C-IL-2 -9 | Equal | 2500 |
| Y31C-IL-2 -10 | Augmented | 600 |
| Y31C-IL-2 -11 | Augmented | 750 |

### EXAMPLE 5

Measuring Reversible Covalent Interactions Between Substrate-Thiolate. and Ligand-Thiolate

A CM5 sensor chip (Biacore-BR-1000-14) is docked in a Biacore 2000 SPR device, and the chip is normalized using a standard Biacore protocol with 70% glycerol. The chip is equilibrated in PBS for approximately 1 hr at a 20 μL/min flow rate. The dextran surface of the chip is activated through an NHS/EDC coupling. This is done using an amine coupling kit (Biacore—BR-100-50) containing N-hydroxysuccinimide (NHS 11.5 mg/mL) and N-ethyl-N′-(3-dimethylaminopropyl) carbodiimide hydrochloride (EDC 75 mg/mL). The two reagents are mixed in a 1:1 ratio and then 35 μL of the mixture are injected at 5 μL/min. The chip is activated one flow cell at a time, and that flow cell is subsequently labeled with protein.

Protein having at least one cysteine is expressed in E. coli or baculovirus-infected insect cells (TN-5B1-4, High Five) and purified using standard procedures. This protein-thiolate is treated with 10 mM βME overnight to reduce the surface cysteine. The βME is removed and the protein-thiolate is placed in 100 mM MES Buffer pH 5.75 (Nap-5 column, Pierce). The protein-thiolate is then concentrated to 100 μM using a Millipore-UFV5BCCOO 5K NMWL membrane, 0.5 mL centrifugal filter and tube.

A sensorgram is then run over the activated flow cell at 2 μL/min of PBS. The protein-thiolate solution is then injected at 2 μL/min for 10 min. After injection one measures the change in RUs by marking the RUs at t=0 and t final and subtracting them. This subtraction gives the total RU change that is related to the total mass of protein successfully coupled to the CM5 surface. This value will later be used to determine stoichiometry of binding. This procedure is then repeated with various cysteine mutants that are immobilized on the adjacent cells. The sensorgram is then altered to flow across all 4 cells on the CM5 chip at 5,L/min PBS. A solution of 1 M ethanolamine hydrochloride-NaOH pH 8.5 (Amine coupling kit, Biacore—BR-100-50) is injected at 5 μL/min for 7 min. The chip is equilibrated overnight in 100 mM Tris pH 8+0.05% azide+1% DMSO at 20 μL/min.

Compounds are synthesized with a reactive disulfide linker (see, for example compound 2t in Example 2). From previous “tethering” experiments compounds that preferentially bind and form a covalent bond with specific cysteine mutants at the active sites of proteins are chosen for these SPR experiments. The compounds are known to form this covalent attachment at relatively high levels of reductant [βME (2-mercaptoethanol, Sigma)], indicating a weak noncovalent binding interaction.

Dilutions series of the compounds are typically performed to produce seven solutions with compound concentrations of 50 mM, 25 mM, 12.5 mM, 6.25 mM, 3.12 mM, 1.56 mM, and 0.78 mM in DMSO, with a final eighth point as a DMSO only baseline. The compounds are then diluted into 100 mM Tris pH 8+0.05% azide+500 μM βME, to final compound concentrations of 500 μM, 250 μM, 125 μM, 62.5 μM, 31.2 μM, 15.6 μM, and 7.8 μM. The compound solutions are injected from lowest concentration to highest at 2 μL/min for 20 min. The SPR software method includes a 1 min wash before injection, the injection and then a 10 min wash after the injection. This particular protocol employs a regeneration step to reduce off the compound that is covalently bound before the next injection. The regeneration is accomplished by injecting 10 mM βME at 2 μL/min for 20 min.

These dilution series are analyzed by BIAevaluation software version 3.0. Sensorgrams are plotted for compound binding to cells of the chip labeled with protein-thiolate. The sensorgrams are modified by subtracting the sensorgrams for the appropriate compound dilutions over cell 1 (a blank cell). This subtraction step removes any RU changes that might occur due to nonspecific interactions between the compound and the chip itself or refractive changes in the buffer. Finally, the eight sensorgrams produced from the compound dilution series are zeroed at the average of their starting RUs. Then the sensorgram produced by injecting DMSO only is subtracted to reveal the final sensorgrams used to determine relative binding.

To determine an effective concentration (EC50SPR) for the compound, the compound concentration is plotted on the X-axis, while the fraction bound at the plateau of the injection is plotted on the Y-axis. The curve is then analyzed via nonlinear regression analysis (Kaleidagraph, Synergy Software). Fraction bound for all of these experiments is determined using the ratio of ligand mass to protein mass and multiplying that by the total RUs produced in the labeling stage of the chip, and dividing the raw RUs by this number. Comparison of EC_{50SPR} values gives a rank order for interaction strength, with smaller values indicating stronger interactions.

This method can be employed in a single point assay format as well. One can choose a standard reductant concentration, and compare various disulfide compounds for percent binding to various protein-thiolates. From this data, a single “fraction bound” value is obtained to rank relative interaction strengths.

### EXAMPLE 6

Ranking Reversible Covalent Interactions Between Substrate-Thiolate and Ligand-Thiolate

In these experiments soluble interleukin-1 receptor, type 1 having a mutation to cysteine, i.e., IL1R-I13C and IL1R-E129C, were immobilized to separate channels of a CM5 chip. The relative ability of 12t, 13t, 14t, and 15t to form a covalent bond with each of the cysteine mutant proteins was measured. The measurement was made at a single disulfide compound concentration and a single reductant concentration of 100 μM and 500 μM, respectively. The ranking is accomplished by measuring the fraction of protein-thiolate on the chip that has made a covalent bond to the various disulfide compounds. Results are shown in Table 4.

TABLE 4

| | | Fraction bound | |
|---|---|---|---|
| | | IL1R- | IL1R- |
| Compound | Structure | I13C | I29C |
| 12t | 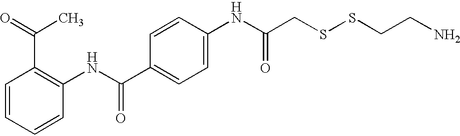 | 0.5 | 0.0 |
| 13t | 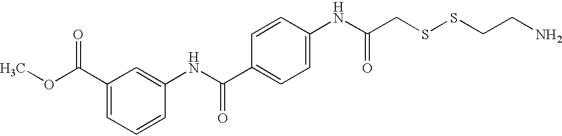 | 0.0 | 0.0 |
| 14t | 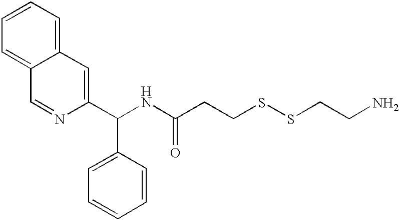 | 0.0 | 0.6 |
| 15t | 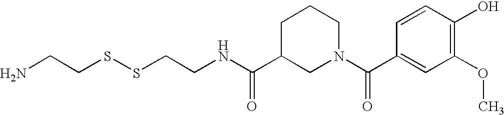 | 0.0 | 0.2 |

As can be seen in Table 4, although 12t and 13t are structurally very similar, 12t can form a covalent bond with the I13C mutant, whereas 13t cannot. Furthermore, compounds 12t, 14t, and 15t have different levels of covalent attachment for each of the two IL-1 R cysteine mutants, showing the importance of the particular location of the thiol on the protein surface.

### EXAMPLE 7

Reductant Titrations to Measure Equilibrium Binding

Proteins having at least one cysteine are coupled to the chip as in Example 5. From previous “tethering” experiments compounds that preferentially bind and form a covalent bond with specific cysteine mutants at the active sites of proteins are chosen for these SPR experiments. The compounds are known to form this covalent attachment at relatively high levels of reductant (βME), indicating a weak noncovalent binding interaction.

Reductant titrations are performed at constant compound concentrations to determine how much reductant can be present while ensuring covalent attachment of the compound. The compound will be in disulfide exchange; use of this particular amount of reductant ensures that the covalent bond is not disrupted, however. For the strength and solubility of the disulfide compounds used, a standard compound concentration of 100 μM was chosen. To perform the βME titration 10 mM compound in DMSO is dissolved in eight different βME concentrations in 100 mM Tris pH 8 buffer+0.05 azide. Final βME concentrations of 2.5 mM, 1.25 mM, 0.625 mM, 0.312 mM, 0.156 mM, 0.078 mM, 0.039 mM and 0.02 mM were used. The 10 mM compound solution is dissolved to a final concentration of 100 μM in the βME dilution series.

The compound/βME solutions are then injected from highest to lowest βME at 2 μL/min for 20 min. The SPR software method includes a 1 min wash before the injection, the injection and then a 10 min wash after the injection. This particular protocol employs a regeneration step to remove the compound that is covalently bound before the next injection. The regeneration is accomplished by injecting 10 mM βME at 2 μL/min for 20 min.

These dilutions series are analyzed by BIAevaluation software version 3.0. Sensorgrams are plotted for compound binding to the cysteine protein labeled cells of the chip. The sensorgrams are modified by subtracting the sensorgrams for the appropriate compound/βME dilutions over cell 1 (a blank cell). This step removes any RU changes that might occur due to non-specific interactions between the compound and the chip itself or refractive changes in the buffer. Finally, the eight sensorgrams produced from the compound/βME dilution series are zeroed at the average of their starting RUs.

To determine μME₅₀ for the compound, the βME concentration is plotted on the X-axis, while the fraction bound at the plateau of the injection is plotted on the Y-axis. The curve is then analyzed via nonlinear regression analysis (Kaleidagraph, Synergy Software). Fraction bound for all of these experiments was determined using the ratio of ligand mass to protein-thiolate mass and multiplying that by the total RUs produced in the labeling stage of the chip, and dividing the raw RUs by this number. Comparison of μME₅₀ values gives a rank order for interaction strength, with a largerμME₅₀ value indicating stronger interactions.

### EXAMPLE 8

Reductant Titration to Measure Disulfide Disruption

Proteins having at least one thiol group are coupled to the chip as in Example 5. From previous “tethering” experiments compounds that preferentially bind and form a covalent bond with specific cysteine mutants at the active sites of proteins are chosen for these SPR experiments. The compounds are known to form this covalent disulfide attachment at relatively high levels of reductant (βME), indicating a weak noncovalent binding interaction.

Reductant titrations are performed at constant compound concentrations; for the strength and solubility of the disulfide compounds used, a standard compound concentration of 100 μM was chosen. Compounds are co-injected in a constant concentration of reductant to ensure covalent attachment to the protein-thiolate. For the compounds chosen, 100 μM βME reductant was present in the compound solutions. 10 mM compound in DMSO is dissolved 100 mM Tris pH 8 buffer+0.05% Azide+100 μM βME. This solution is injected first to ensure covalent attachment of the disulfide compound. After equilibrium is reached, βME is injected to release the compound. Solutions with final βME concentrations of 10 mM, 5 mM, 2.5 mM, 1.25 mM, 0.625 mM, 0.312 mM, 0.156 mM and 0.78 mM were prepared in 100 mM Tris pH 8+0.05% Azide.

The compound solutions are then injected at 2 μL/min for 20 min one at a time with an alternating injection of the βME dilution series. The SPR software method includes a 1 min wash before injection, the injection and then a 10 min wash after the injection. This particular protocol employs a regeneration step before the next compound injection to remove any compound that is covalently bound before the next injection. The regeneration is accomplished by injecting 10 mM βME at 2 μL/min for 20 min. In summary, the injections are performed in the following order. First 100 μM compound in 100 μM βME is injected, next the βME dilution point is injected, and finally 3-10 mM βME is injected to ensure all compound is reduced off the cysteine.

These dilution series are analyzed by BIAevaluation software version 3.0. Sensorgrams are plotted for compound binding to cells of the chip labeled with protein-thiolate. The sensorgrams are modified by subtracting the sensorgrams for the appropriate compound/βME dilutions over cell 1 (a blank cell). This step removes any RU changes that might occur due to nonspecific interactions between the compound and the chip itself or refractive changes in the buffer. Finally the eight sensorgrams produced from the compound/βME dilution series are zeroed at the average of their starting RUs.

To determine βME_{50SPR} for the compounds, the βME concentration is plotted on the X-axis, while the fraction bound at the end of the injection (once the new equilibrium is reached) is plotted on the Y-axis. The curve is then analyzed via nonlinear regression analysis (Kaleidagraph, Synergy Software). Fraction bound for all of these experiments was determined using the ratio of ligand mass to protein-thiolate mass and multiplying that by the total RUs produced in the labeling stage of the chip, and dividing the raw RUs by this number.

### EXAMPLE 9

Double Ligand SPR

A CM5 sensor chip (Biacore-BR-1000-14) is docked in a Biacore 2000 SPR device and normalized using a standard Biacore protocol with 70% glycerol. The chip is next equilibrated in PBS for approximately 1 hr at a 20 μL/min flow rate. The dextran surface of the chip is activated through an NHS/EDC coupling. This coupling is done using an amine coupling kit (Biacore—BR-100-50) containing N-hydroxysuccinimide (NHS 11.5 mg/mL) and N-ethyl-N′-(3-dimethylaminopropyl) carbodiimide hydrochloride (EDC 75 mg/mL). First, the two reagents are mixed in a 1:1 ratio and then 35 μL of the mixture are injected at 5 μL/min. The chip is activated one flow cell at a time, and that flow cell is subsequently labeled with protein.

IL-2 is expressed in E. coli cells and purified using standard procedures. Protein is then concentrated to 100 μM and the buffer is exchanged to 100 mM MES pH 5.75 (Nap-5 column, Pierce). A sensorgram is then run over the activated flow cell at 2,L/min of PBS. The IL-2 solution is then injected at 2 μL/min for 10 min. After injection the change in RUs is measured by marking the RUs at t=0 and t=final and subtracting. This gives the total RU change, which is related to the total mass of protein successfully coupled to the CM5 surface. The total mass will later be used to determine stoichiometry of binding. This procedure is then repeated with another arbitrary protein to create a measure for nonspecific binding. The arbitrary protein is immobilized on an adjacent cell. A protein of comparable molecular weight is chosen, so RU values will be similar. The sensorgram is then altered to flow across all four cells on the CM5 chip at 5 μL/min PBS. A solution of 1 M ethanolamine hydrochloride-NaOH pH 8.5 (Amine coupling kit, Biacore-BR-100-50) is injected at 5 μL/min for 7 min. Proteins are immobilized at densities of 100-160 fmol/mm². The chip is equilibrated overnight in PBS+0.05% azide+2% DMSO at 20 μL/min.

In this experiment, variable concentrations of a compound are tested at a constant concentration of another compound. The compound held constant is 2c, for example (structure shown in Example 2). To determine appropriate constant concentrations for 2c, a compound titration is first performed. Dilution series of the compounds are typically performed to produce seven solutions having final compound concentrations of 30 mM, 10 mM, 3.3 mM, 1.1 mM, 0.37 mM, 0.12 mM, and 0.041 mM; an eighth solution is a DMSO-only baseline. The compounds are then diluted into PBS+0.05% azide to final concentrations of 300 μM, 100 μM, 33 μM, 11 μM, 3.7 μM, 1.2 μM, 0.41 μM. The compound solutions are injected from lowest concentration to highest at 40 μL/min for 1 min. The SPR software method includes a 1 min wash before injection, the injection and then a 3 min wash after the injection.

These dilution series are analyzed by BIAevaluation software version 3.0. Sensorgrams are plotted for compound binding to the IL-2 labeled cell of the chip. The sensorgrams are modified by subtracting the sensorgrams for the appropriate compound dilutions over cell 1 (a blank cell). This subtraction step removes any RU changes that might occur due to non-specific interactions between the compound and the chip itself or refractive changes in the buffer. Finally, the eight sensorgrams produced from the compound dilution series are zeroed at the average of their starting RUs. Then the sensorgram produced by injecting DMSO only is subtracted to reveal the final sensorgrams used to determine relative binding.

To determine K_{dSPR} for the compound, the compound concentration is plotted on the X-axis, while the fraction bound at the plateau of the injection is plotted on the Y-axis. The curve is then analyzed via non-linear regression analysis (Kaleidagraph, Synergy Software). From this data a saturating concentration of compound can be chosen. The concentration chosen is where the RUs remain constant at the top of the binding curve and thus the IL-2:2c ratio is 1. For 2c, this concentration is 100 μM.

A second compound is then run, this time in both the presence and absence of 2c. Dilution series of the second compound are typically performed to produce seven solutions having compound concentrations of 50 mM, 25 mM, 12.5 mM, 6.2 mM, 3.1 mM, 1.5 mM, 0.75 mM in DMSO, and an eighth solution is used as a DMSO-only baseline. The second compound is then diluted into PBS+0.05% azide or PBS+0.05% azide+100 μM 2c to final compound concentrations of 500 μM, 250 μM, 125 μM, 62 μM, 31 μM, 15 μM, 7.5 μM. The solutions of the second compound are injected from lowest concentration to highest at 40 μL/min for 1 min. The SPR software method includes a 1 min wash before injection, the injection and then a 3 min wash after the injection.

These dilution series are analyzed by BIAevaluation software version 3.0. Sensorgrams are plotted for compound binding to the IL-2 labeled cell of the chip. The sensorgrams are modified by subtracting the sensorgrams for the appropriate compound dilutions over cell 1 (a blank cell). This step removes any RU changes that might occur due to non-specific interactions between the compound and the chip itself or refractive changes in the buffer. Finally, the eight sensorgrams produced from the compound dilution series are zeroed at the average of their starting RUs. Then the sensorgram produced by injecting DMSO only or DMSO+100 μM 2c is subtracted to reveal the final sensorgrams used to determine relative binding. The baseline is set by the RUs created by the binding of 2c—thus all additional RUs seen by subtracting this baseline are assumed to be due to direct binding of the titrated ligand. In the case of an noncooperative interaction one may observe no additional RUs, or a loss of RUs as 2c cannot bind in the presence of increasing concentrations of this new compound (the decrease is due to the fact that although one compound is always bound there is a discrepancy in their masses and thus a change in the RUs observed). In the case of positive cooperativity, additional RUs will be observed.

The K_{dSPR} of the second compound in the absence of 2c is obtained by plotting the concentration of the second compound on the X-axis, and plotting the fraction bound of the second compound in the absence of 2c on the Y-axis. The K_{dSPR} of the second compound in the presence of 2c is obtained by plotting the concentration of the second compound on the X-axis, and plotting the fraction bound of the second compound in the presence of 2c on the Y-axis. Each of the curves is then analyzed via nonlinear regression analysis (Kaleidagraph, Synergy Software) and the K_{dSPR} values can be compared.

### EXAMPLE 10

Competitive Binding of Compounds to Immobilized Interleukin-2

Interleukin-2 (“IL-2”) was immobilized onto the surface and various concentrations (0-500 μM) of 1 were allowed to flow over the immobilized IL-2.

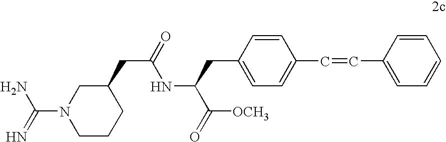

FIG. 7 shows the changes in refractive index over time. As also shown by FIG. 7, the apparent K_{d} is about 175 μM with 1:1 binding occurring at 20 RU (refractive index units).

The same experiment was performed in the presence of a constant concentration of a second compound (50 μM of 2c). The RU value of 0 is the value of the refractive index observed for the immobilized IL-2 in the presence of 50,M of 2c. As can be seen in FIG. 8, the addition of increasing concentrations of compound 10 yields a concomitant decrease in the refractive index values. The decreases in refractive index values indicate that 1 is inhibiting the binding of 2c to interleukin-2.

These data can be converted from plateau refractive index values into % bound values, and plotted as a function of the concentration of 1. The resulting dose-response plot is shown in FIG. 9. Shown is the K_{d} for 1 binding IL-2, which is 210 μM, along with the IC₅₀ of 1 inhibiting the binding of 2c, which is 185 μM.

### EXAMPLE 11

Cooperative Binding of Compounds to Immobilized Interleukin-2

The apparent K_{d}S of 2c, 16, and 3c in SPR experiments are 10 μM, 5 μM and greater than 500 μM respectively. The structures of 2c and 3c are shown in Example 2 and the structure of 16 is shown below.

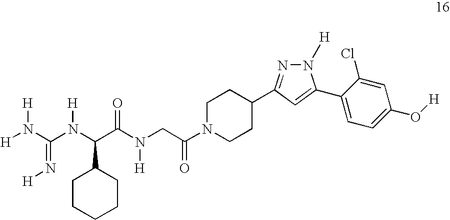

In this experiment, either 2c or 16 was held at a fixed concentration, and the concentration of 3c was variable. FIG. 10 shows the change in plateau refractive index values (converted into % bound) as a function of the concentration of 3c. As it can be seen, an increase in plateau value is seen for the binding of 3c to interleukin-2 in the presence of 50,M of 2c over that seen in the absence of 50 μM of 2c. This increase in refractive index values indicates that 3c and 2c bind to interleukin-2 in a cooperative manner. Likewise, there is an increase in the refractive index values for 3c binding to interleukin-2 in the presence of 16, relative to those in the absence of 16. This increase in refractive index values indicates that 3c and 16 also bind to interleukin-2 in a cooperative manner. Table 5 shows the results of compounds binding cooperatively to immobilized IL-2.

TABLE 5

| | | K_{d,SPR,} □M | | |
|---|---|---|---|---|
| Compound | Structure | IL-2 | IL-2 + 1 | IL-2 + 2c |
| 3c | 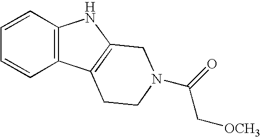 | >500 | 230 | 206 |
| 4c | 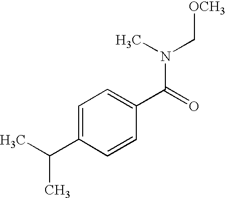 | Inactive | 240 | NA |
| 17 | 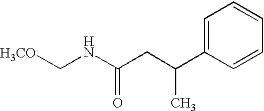 | 500 | 267 | 293 |
| 18 | 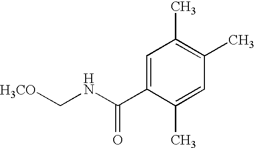 | >1000 | >1000 | 141 |
| 19 | 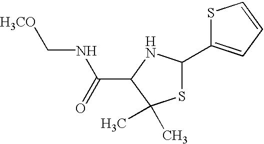 | 200 | 80 | NA |
| 20 | 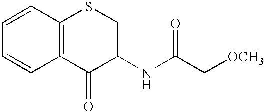 | Inactive | 200 | NA |

### EXAMPLE 12

Protocol for Measuring Competitive and Cooperative Noncovalent Binding of Immobilized Ligand and Free Ligand to a Substrate

A CM5 sensor chip (Biacore-BR-1000-14) is docked in a Biacore 2000 SPR device. The chip is then normalized using standard Biacore protocol with 70% glycerol. The chip is next equilibrated in PBS for approximately 1 hr at 20,L/min flow rate. The dextran surface of the chip is activated through an NHS/EDC coupling. This coupling is done using an amine coupling kit (Biacore—BR-100-50) containing N-hydroxysuccinimide (NHS 11.5 mg/mL) and N-ethyl-N′-(3-dimethylaminopropyl) carbodiimide hydrochloride (EDC 75 mg/mL). The two reagents are mixed in a 1:1 ratio and 35 μL of the mixture are injected at 5 μL/min. The activated chip is then modified with a cysteine reactive group, to enable labeling of the CM5 chip with cysteine chemistry. A solution of 100 μM MTSL in PBS is injected at 2 μL/min for 10 min over the activated cell.

A peptide that is a nanomolar inhibitor for the protein of interest was synthesized at Synpep, to include a cysteine residue at the N-terminus. This peptide was brought up to a 100 μM solution in PBS. This solution is then injected over the activated cell at 2 μL/min for 10 min. After injection one measures the change in RUs by marking the RUs at t=0 and t=final and subtracting. This gives the total RU change, which is related to the total mass of peptide successfully coupled to the CM5 surface. The sensorgram is then altered to flow across all four cells on the CM5 chip at 5 μL/min PBS. A solution of 1 M ethanolamine hydrochloride-NaOH pH 8.5 (Amine coupling kit, Biacore—BR-100-50) is injected at 5 mL/min for 7 min. The peptide is immobilized at a density of 40 fmol/mm². The chip is equilibrated overnight in PBS+0.05% azide at 20 μL/min.

IL-1 R is expressed in baculovirus-infected TN-5B1-4 insect cells (High Five) and purified using standard procedures. To determine an appropriate constant concentration of protein to use, a protein titration is first performed. A dilution series of the protein is typically performed to produce seven protein solutions having final concentrations of from 300 nM, 100 nM, 33 nM, 11 nM, 3.7 nM, 1.2 nM, and 0.41 nM in PBS+0.05% azide, and an eighth solution is used as a PBS-only baseline. The protein solutions are injected from lowest concentration to highest at 40 μL/min for 1 min. The SPR software method includes a 1 min wash before injection, the injection and then a 3 min wash after the injection.

These dilutions series are analyzed by BIAevaluation software version 3.0. Sensorgrams are plotted for protein binding to the immobilized peptide cell. The sensorgrams are modified by subtracting the sensorgrams for the appropriate protein dilutions over cell 1 (a blank cell). This subtraction step removes any RU changes that might occur due to nonspecific interactions between the protein and the chip itself or refractive changes in the buffer. Finally, the eight sensorgrams produced from the protein dilution series are zeroed at the average of their starting RUs. Then the sensorgram produced by injecting PBS only is subtracted to reveal the final sensorgrams used to determine relative binding.

To determine K_{dSPR} for the protein, the protein concentration is plotted on the X-axis, while the fraction bound at the plateau of the injection is plotted on the Y-axis. The curve is then analyzed via nonlinear regression analysis (Kaleidagraph, Synergy Software). From this data, a subsaturating concentration of protein can be chosen. One wants to keep protein constant and non-saturated to be assured of not having an excess of protein in solution. For this protein, 30 nM was chosen.

A new set of ligands is then run, in both the presence and absence of 30 nM protein. Dilution series of the compounds are typically performed to produce seven solutions containing compound concentrations of 50 mM, 25 mM, 12.5 mM, 6.2 mM, 3.1 mM, 1.6 mM, and 0.78 mM in DMSO, and an eighth solution is used as DMSO-only baseline. The compounds are then diluted into PBS+0.05% azide or PBS+0.05% azide+30 nM protein, to final concentrations of 500 μM, 250 μM, 125 μM, 62 μM, 31 μM, 16 μM, and 7.8 μM. The running buffer on the Biacore is then exchanged for PBS+0.05% azide+1% DMSO. The dilution series are injected from lowest concentrations of compound to the highest. The injections are performed at 40 μL/min for 1 min. The SPR software method includes a 1 min wash before injection, the injection and then a 3 min wash after the injection.

These dilution series are analyzed by BIAevaluation software version 3.0. Sensorgrams are plotted for protein binding to the immobilized peptide cell. The sensorgrams are modified by subtracting the sensorgrams for the appropriate compound dilutions over cell 1 (a blank cell). This step removes any RU changes that might occur due to non-specific interactions between the compound and the chip itself or refractive changes in the buffer. Finally the eight sensorgrams produced from the compound dilution series are zeroed at the average of their starting RUs. To determine IC50SPR for the compound, the compound concentration is plotted on the X-axis, while the fraction bound at the plateau of the injection is plotted on the Y-axis. The curve is then analyzed via nonlinear regression analysis (Kaleidagraph, Synergy Software).

### EXAMPLE 13

Competitive Binding to an Immobilized Peptide Ligand

A peptide ligand of the human interleukin-1 receptor type 1 (II-1 R) was immobilized to a surface, and various small molecule ligands were tested to examine whether they compete for the same binding site on II-1 R. The peptide has the following sequence.

CETPFTWEESNAYYWQPYALPL (SEQ ID NO:1)

First, soluble human II-1 R was allowed to flow over the surface containing the immobilized peptide ligand, and the refractive index was measured. Next a small molecule ligand was added to the 11-1 R solution, and the resulting refractive index was measured. Shown in Table 6 are IC₅₀ values for the inhibition of binding of IL-1 R to the immobilized peptide by several small molecule compounds.

TABLE 6

| Compound | Structure | IC_{50SPR}, μM |
|---|---|---|
| 21 | 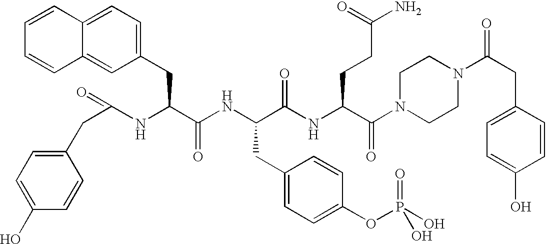 | 10.5 |
| 22 | 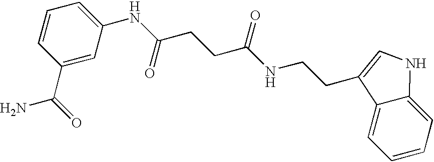 | Inactive |
| 23 | 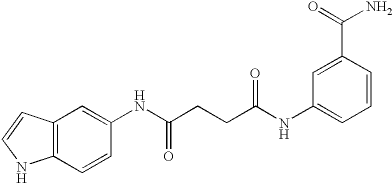 | 560 |
| 24 | 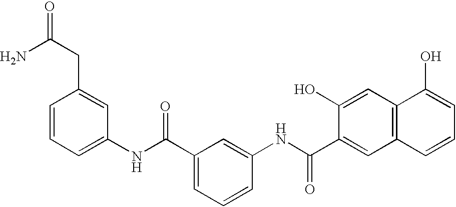 | 95 |
| 25 | 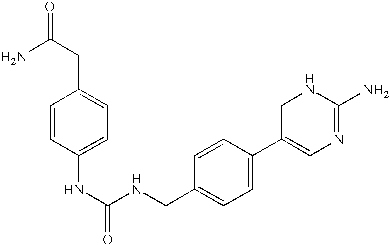 | 240 |

What is claimed is:

1. A method comprising:
a) measuring by SPR a first extent of interaction between a first ligand and a substrate in the presence of a second ligand, wherein the substrate possesses a first chemically reactive group and the second ligand possesses a second chemically reactive group and the second ligand is capable of forming a reversibly covalent substrate-ligand complex with the substrate, and wherein the first ligand or the substrate is immobilized to a surface,
b) measuring by SPR a second extent of interaction between the first ligand and the substrate in the absence of the second ligand, and
c) comparing the first extent of interaction with the second extent of interaction.

2. The method of claim 1 wherein the first ligand and the second ligand are binding cooperatively to the substrate and the first extent of interaction is greater than the second extent of interaction.

3. The method of claim 1 wherein the first ligand and the second ligand are binding simultaneously to the substrate, and the first extent of interaction is equal to the second extent of interaction.

4. The method of claim 1 wherein the first ligand and the second ligand are binding competitively to the substrate and the first extent of interaction is less than the second extent of interaction.

5. The method of claim 1 wherein the first extent of binding and the second extent of binding are measured in parallel.

6. The method of claim 1 wherein the substrate is a substrate-thiolate, the first chemically reactive group is a first thiol group, the second chemically reactive group is a second thiol group, and wherein the second ligand is capable of forming a substrate-SS—ligand complex with the substrate-thiolate.

7. The method of claim 6 wherein the first ligand is immobilized to the surface.

8. The method of claim 7 wherein the first ligand is a ligand-thiolate capable of forming an immobilized ligand-SS—substrate complex with the substrate-thiolate.

9. The method of claim 7 wherein the first extent of binding is measured under conditions where the substrate-SS—ligand complex has formed.

10. The method of claim 6 wherein the substrate-thiolate is immobilized to the surface.

11. The method of claim 10 wherein the first extent of binding is measured under conditions where an immobilized substrate-SS—ligand complex between the substrate-thiolate and the second ligand has formed.

12. A method comprising:
a) measuring by SPR a first extent of interaction between a first ligand and a substrate-thiolate in the presence of a second ligand, wherein the substrate-thiolate possesses a first thiol group, and the second ligand possesses a second thiol group, and the substrate-thiolate and the second ligand are capable of forming a substrate-SS—ligand complex, and wherein the first ligand or the substrate-thiolate is immobilized to a surface,
b) measuring by SPR a second extent of interaction between the first ligand and the substrate-thiolate in the absence of the second ligand, and
c) comparing the first extent of interaction with the second extent of interaction.

13. The method of claim 1 or claim 12 wherein one of the first ligand and second ligand is a peptide and the other is a small molecule of less than 2000 Da.

14. The method of claim 1 or claim 12 wherein the first ligand and the second ligand are each a small molecule of less that 2000 Da.

15. A method comprising:
a) measuring by SPR a calibration corresponding to an extent of formation of a reversible covalent bond between a substrate and a ligand in the presence of a reversing agent at a first concentration wherein the first concentration is sufficient to disrupt the reversible covalent bond and wherein the substrate or the ligand is immobilized,
b) measuring by SPR a plurality of test values wherein each value corresponds to an extent of formation of the reversible covalent bond between the substrate and the ligand at a concentration of a reversing agent that is less than the first concentration, and
c) correlating the extent of formation of the reversible covalent bond as a function of the concentration of the reversing agent.

16. The method of claim 15 wherein the ligand is a small molecule of less than 2000 Da.

17. The method of claim 1, 12, or 15 wherein the substrate is a protein.
